SEQUENCE LISTING (6 sequences)

SEQ ID NO: 1 - RNA, 20 nt, synthetic construct
  misc_feature 1..20: note = Fah crRNA1
  source 1..20: mol_type = other RNA
tctactcttctcggcagtcg

SEQ ID NO: 2 - RNA, 20 nt, synthetic construct
  misc_feature 1..20: note = Fah crRNA2
  source 1..20: mol_type = other RNA
gtggtcgaggctagaactag

SEQ ID NO: 3 - DNA, 20 bp, synthetic construct
  misc_feature 1..20: note = human mitochondrial tRNA-Leu(UUR) forward primer
  source 1..20: mol_type = other DNA
cacccaagaacagggtttgt

SEQ ID NO: 4 - DNA, 20 bp, synthetic construct
  misc_feature 1..20: note = human mitochondrial tRNA-Leu(UUR) reverse primer
  source 1..20: mol_type = other DNA
tggccatgggtatgttgtta

SEQ ID NO: 5 - DNA, 20 bp, synthetic construct
  misc_feature 1..20: note = mouse nuclear 18s rRNA forward primer
  source 1..20: mol_type = other DNA
tagagggacaagtggcgttc

SEQ ID NO: 6 - DNA, 18 bp, synthetic construct
  misc_feature 1..18: note = mouse nuclear 18s rRNA reverse primer
  source 1..18: mol_type = other DNA
cgctgagccagtcagtgt

(19) United States
(12) Patent Application Publication
(10) Pub. No.: US 2025/0195684 A1
(43) Pub. Date: Jun. 19, 2025

(54) DELIVERY METHODS USING PLATELETS

(71) Applicant: Children's Hospital Medical Center, Cincinnati, OH (US)

(72) Inventors: Takanori Takebe, Cincinnati, OH (US); Masaki Kimura, Cincinnati, OH (US); Andrew Dunn, Worthington, OH (US); Takuma Iguchi, Tokyo (JP)

(73) Assignee: Children's Hospital Medical Center, Cincinnati, OH (US)

(21) Appl. No.: 18/847,004

(22) PCT Filed: Mar. 22, 2023
(86) PCT No.: PCT/US2023/064831

§ 371 Date: Sep. 13, 2024

Related U.S. Application Data
(60) Provisional application No. 63/323,280, filed on Mar. 24, 2022.

(51) Int. Cl. A61K 47/69; A61K 31/7088; A61K 35/19; A61K 38/46; A61K 47/64; A61K 48/00; C12N 5/078

(52) U.S. Cl. CPC A61K 47/6901; A61K 31/7088; A61K 35/19; A61K 38/465; A61K 47/64; A61K 48/0033; A61K 48/005; C12N 5/0644; C12N 2506/45

(57) ABSTRACT

Disclosed herein are methods of transferring exogenous material using platelets. The methods comprise administering the platelets with the exogenous material to a recipient. The platelets may be obtained by directly contacting the platelets, or contacting megakaryocytes that produce the platelets.

### CROSS-REFERENCE TO RELATED APPLICATIONS

This application claims priority to and benefit of U.S. Provisional Patent Application No. 63/323,280, filed Mar. 24, 2022.

### REFERENCE TO SEQUENCE LISTING

The present application is being filed along with a Sequence Listing in electronic format. The Sequence Listing is provided as a file entitled CHMC63029WO.xml, which was created and last modified on Mar. 20, 2023, which is 8,514 bytes in size. The contents of the Sequence Listing in electronic format is hereby expressly incorporated by reference in its entirety.

### FIELD OF THE INVENTION

Aspects of the present disclosure relate generally to methods of transferring exogenous material and/or a derivative thereof to cells using platelets as the delivery mechanism.

### BACKGROUND

As knowledge of genetics increases, more and more diseases are recognized as genetic disorders. Even though most genetic diseases are rare, the sheer number of these disorders (˜8,000 known at present) leads to a total of 6-8% of the human population being affected. Therefore, gene therapy research has increased dramatically in demand in recent times. On the other hand, nucleic acid-based therapies, particularly vaccines, have been groundbreaking and still hold great potential in the treatment of cancer or the prevention of infectious disease pandemics. The key to implement these therapies, especially mRNA-based therapies, in clinical practice relies on a robust delivery system.

Currently, the two major in vivo gene delivery systems are viral-based and nanoparticle-based gene delivery systems. However, the problems of low-expression efficiency and safety concerns still largely remain unsolved. Reports show that unwanted immune responses can be triggered by these gene delivery methods, which can cause very severe secondary consequences. There is a lasting need for alternative methods of delivering biological material, such as nucleic acids and proteins, particularly to humans and other animals in a clinical setting.

### SUMMARY

Disclosed herein are methods of preparing a platelet comprising exogenous material and/or a derivative thereof. In some embodiments, the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof. In some embodiments, the nucleic acid comprises DNA, or RNA, or both. In some embodiments, the RNA comprises mRNA, ncRNA, asRNA, IncRNA, miRNA, piRNA, siRNA, shRNA, gRNA, or any other type of RNA known in the art. In some embodiments, the nucleic acid encodes for one or more viral, bacterial, or protozoal protein. In some embodiments, the one or more viral, bacterial, or protozoal protein is immunogenic, for example, in humans. In some embodiments, the nucleic acid encodes for a gene editing protein, for example, the gene editing proteins disclosed herein. In some embodiments, the gene editing protein comprises a zinc finger nuclease, TALEN, nuclease, CRISPR nuclease, Cas9, or a base editor, or any combination thereof. In some embodiments, the methods comprise transfecting a platelet with the exogenous material and/or the derivative thereof. In some embodiments, the platelet is isolated from a donor subject. In some embodiments, the methods comprise transfecting a megakaryocyte with the exogenous material and/or the derivative thereof. In some embodiments, where the desired exogenous material and/or the derivative thereof comprises a protein, the methods comprise transfecting a megakaryocyte with exogenous material and/or the derivative thereof comprising a nucleic acid encoding for the protein. In some embodiments, the megakaryocyte is isolated from a donor subject. In some embodiments, the donor subject has been provided with a megakaryocyte stimulator, for example, the megakaryocyte stimulators disclosed herein. In some embodiments, the megakaryocyte stimulator is thrombopoietin romiplostim, eltrombopag, avatrombopag, lusutrombopag, or any combination thereof. In some embodiments, the platelet or megakaryocyte, or both, is transfected. In some embodiments, the platelet comprising exogenous material and/or the derivative thereof is activated with a platelet activator, for example, the platelet activators described herein. In some embodiments, the platelet activator is thrombin ADP, calcium, thromboxane A2 (TXA2), platelet activating factor (PAF), cathepsin G, von Willebrand factor, collagen, fibrinogen, or laminin, or any combination thereof. In some embodiments, the platelet further comprises a cell surface modification that enhances selectivity to a specific cell type or types. In some embodiments, the cell surface modification comprises modifications to the surface glycans of the platelet and/or an exogenous targeting ligand that is selective for the specific cell type or types.

Also disclosed herein are methods of delivering exogenous material and/or the derivative thereof to a recipient cell or subject. The methods comprise administering a platelet comprising exogenous material and/or the derivative thereof to the recipient cell or subject. In some embodiments, the platelet comprising exogenous material and/or the derivative thereof is prepared by any one of the methods disclosed herein. In some embodiments, the platelet comprising exogenous material and/or the derivative thereof is administered in vivo to the recipient cell or subject. In some embodiments, the administration is selected from parenterally, intramuscularly, intraperitoneally, intravenously, subcutaneously, trans-hepatically, intra-vascularly, intra-hepatically, intra-portally, intra-splenically, and intradermally. In some embodiments, the administration is done by ultrasound-guided injection.

In some embodiments, the platelets or megakaryocytes, or both, are isolated from a donor subject. In some embodiments, the donor subject is a mammal. In some embodiments, the donor subject is a human. In some embodiments, the donor platelets are isolated from the donor subject by platelet apheresis. In some embodiments, the donor subject is first provided with a megakaryocyte stimulator, for example, the megakaryocyte stimulators disclosed herein. In some embodiments, the recipient subject is a mammal. In some embodiments, the recipient subject is a human. In some embodiments, both the donor subject and recipient subject are human, and the administration of the platelet comprising exogenous material and/or the derivative thereof is allogeneic. In some embodiments, the donor subject and recipient subject is the same individual, and the administration of the platelet comprising exogenous material and/or the derivative thereof is autologous. In some embodiments, the platelet is obtained or derived from the recipient subject. In some embodiments, the platelet is obtained or derived from a megakaryocyte obtained or derived from the recipient subject. In some embodiments, the platelet and/or megakaryocyte is obtained or derived from pluripotent stem cells obtained or derived from the recipient subject. In some embodiments, the recipient subject has a disease, had a disease previously, or is at risk of contracting a disease. In some embodiments, the disease is a genetic disease. In some embodiments, the disease is a metabolic disease. In some embodiments, the platelet comprising exogenous material and/or the derivative thereof is administered to the recipient subject as a therapeutic. In some embodiments, the platelet comprising exogenous material and/or the derivative thereof is administered as a platelet composition.

Also disclosed herein is the use of a platelet comprising exogenous material and/or the derivative thereof in a subject as a therapeutic. In some embodiments, the platelet comprising exogenous material and/or the derivative thereof is used to induce immune protection against a virus, bacteria, or protozoan in a subject. In some embodiments, the platelet comprising exogenous material and/or the derivative thereof is used to edit a gene in a subject. In some embodiments, the subject is a mammal. In some embodiments, the subject is a human.

Embodiments of the present disclosure provided herein are described by way of the following numbered alternatives:
1. A method of preparing a platelet comprising exogenous material and/or a derivative thereof, comprising:
a) contacting the platelet with the exogenous material and/or the derivative thereof, or
b) contacting a megakaryocyte with the exogenous material and/or the derivative thereof and allowing the megakaryocyte to produce the platelet comprising the exogenous material and/or the derivative thereof;
thereby preparing the platelet comprising the exogenous material and/or the derivative thereof.
2. The method of alternative 1, wherein the exogenous material and/or the derivative thereof is biological material.
3. The method of alternative 1 or 2, wherein the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof.
4. The method of alternative 3, wherein the small molecule is a therapeutic small molecule.
5. The method of alternative 3 or 4, wherein the nucleic acid comprises DNA or RNA, or both.
6. The method of alternative 5, wherein the RNA is messenger RNA (mRNA), noncoding RNA (ncRNA), antisense RNA (asRNA), long noncoding RNA (lncRNA), microRNA (miRNA), Piwi-interacting RNA (piRNA), small interfering RNA (siRNA), short hairpin RNA (shRNA), or guide RNA (gRNA), or any combination thereof.
7. The method of any one of alternatives 3-6, wherein the nucleic acid encodes for one or more viral, bacterial, or protozoal proteins or a portion thereof, or the protein is the one or more viral, bacterial, or protozoal proteins or portion thereof, wherein the one or more viral, bacterial, or protozoal proteins or portion thereof are immunogenic.
8. The method of any one of alternatives 3-6, wherein the nucleic acid encodes for a gene editing protein or the protein is the gene editing protein.
9. The method of alternative 8, wherein the gene editing protein comprises a zinc finger nuclease, TALEN, nuclease, CRISPR nuclease, Cas9, or a base editor, and optionally, wherein the exogenous material and/or the derivative thereof further comprises a gRNA or a nucleic acid encoding for the gRNA.
10. The method of any one of alternatives 1-9, wherein contacting the platelet with the exogenous material and/or the derivative thereof or contacting the megakaryocyte with the exogenous material and/or the derivative thereof comprises transfection.

11. The method of alternative 10, wherein the transfection comprises electroporation, lipofection, or viral transduction.
12. The method of any one of alternatives 1-11, wherein for option b):
i) the exogenous material and/or the derivative thereof comprised by the platelet is the exogenous material and/or the derivative thereof contacted with the megakaryocyte; or
ii) the exogenous material and/or the derivative thereof comprised by the platelet is derived from the exogenous material and/or the derivative thereof contacted with the megakaryocyte.
13. The method of alternative 12, wherein the exogenous material and/or the derivative thereof comprised by the platelet comprises a protein and the exogenous material and/or the derivative thereof contacted with the megakaryocyte comprises a nucleic acid, wherein the protein is expressed from the nucleic acid.
14. The method of alternative 12, wherein the exogenous material and/or the derivative thereof comprised by the platelet comprises a first nucleic acid and the exogenous material and/or the derivative thereof contacted with the megakaryocyte comprises a second nucleic acid, wherein the first nucleic acid is the second nucleic acid or is derived from the second nucleic acid.
15. The method of alternative 12, wherein the exogenous material and/or the derivative thereof comprised by the platelet comprises a first protein and the exogenous material and/or the derivative thereof contacted with the megakaryocyte comprises a second protein, wherein the first protein is the second protein or a post-translationally modified variant of the second protein.
16. The method of any one of alternatives 1-15, wherein the platelet further comprises a cell surface modification that enhances selectivity to a specific recipient cell type or types.
17. The method of alternative 16, wherein the cell surface modification comprises a modification to the surface glycans of the platelet.
18. The method of alternative 16 or 17, wherein the cell surface modification comprises desialylation of the surface glycans of the platelet.
19. The method of any one of alternatives 16-18, wherein the cell surface modification comprises an exogenous targeting ligand on or associated with the cell surface of the platelet, wherein the exogenous targeting ligand selective for the specific recipient cell type or types.
20. The method of alternative 19, wherein the exogenous targeting ligand is biotinylated and is bound to a streptavidin complex, which is further bound to a biotinylated platelet-specific ligand that is bound to the platelet.
21. The method of alternative 19, wherein the exogenous targeting ligand comprises a transmembrane component and an extracellular component.
22. The method of alternative 21, wherein the transmembrane component is a protein normally found in platelets.
23. The method of alternative 22, wherein the protein normally found in platelets comprises CD41, CD42a, CD42b, CD61, CD9, CD29, CD31, CD36, CD62P, CD63, CD107a, CD154, glycoprotein VI, integrin αIIbβ3, or any combination thereof.
24. The method of any one of alternatives 19-23, wherein the exogenous targeting ligand is expressed by the platelet and/or the megakaryocyte from which the platelet is produced.
25. The method of any one of alternatives 1-24, further comprising stimulating the megakaryocyte with a megakaryocyte stimulator.
26. The method of alternative 25, wherein the megakaryocyte stimulator is thrombopoietin, romiplostim, eltrombopag, avatrombopag, lusutrombopag, or any combination thereof.
27. The method of any one of alternatives 1-26, further comprising activating the platelet comprising the exogenous material and/or the derivative thereof with a platelet activator.
28. The method of alternative 27, wherein the platelet activator is thrombin, ADP, calcium, thromboxane A2 (TXA2), platelet activating factor (PAF), cathepsin G, von Willebrand factor, collagen, fibrinogen, or laminin, or any combination thereof.
29. A method of delivering exogenous material and/or a derivative thereof to a recipient cell, comprising contacting a platelet comprising the exogenous material and/or the derivative thereof with the recipient cell, thereby delivering the exogenous material and/or the derivative thereof to the recipient cell.
30. The method of alternative 29, wherein the platelet is produced by any one of methods of alternatives 1-28.
31. The method of alternative 29 or 30, wherein the platelet comprises a gene editing protein and the gene editing protein modifies the genome of the recipient cell.
32. The method of alternative 31, wherein the gene editing protein modifies the genome of a plurality of recipient cells, optionally wherein the plurality of recipient cells is part of an organ or tissue.
33. The method of any one of alternatives 29-32, wherein the recipient cell(s) is/are characterized by a disease and the modification to the genome of the recipient cell(s) treats the disease.
34. The method of alternative 33, wherein the disease is tyrosinemia type 1 and the modification to the genome comprises a modification to the fumarylacetoacetate hydrolase (Fah) gene.
35. The method of alternative 33, wherein the disease is ornithine transcarbamylase deficiency and the modification to the genome comprises a modification to the ornithine transcarbamylase (OTC) gene.
36. The method of alternative 33, wherein the disease is citrullinemia type 1 (CTLN1) and the modification to the genome comprises a modification to the argininosuccinate synthetase (ASS1) gene.
37. The method of alternative 33, wherein the disease is adult-onset type II citrullinemia (CTLN2) and the modification to the genome comprises a modification to the citrin (solute carrier family 25, member 13; SLC25A13) gene.
38. The method of any one of alternatives 29-37, wherein said contacting occurs in vitro.
39. The method of any one of alternatives 29-38, wherein said contacting occurs ex vivo.
40. The method of alternative 39, wherein said recipient cell(s) are obtained or derived from a recipient subject, and further comprising administering the recipient cell(s) to the recipient subject following the step of contacting the platelet with the recipient cell(s).
41. The method of any one of alternatives 29-37, wherein said contacting occurs in vivo.
42. The method of alternative 41, wherein said recipient cell(s) is/are in a recipient subject, and said contacting comprises administering said platelet to said recipient subject.
43. The method of alternative 40 or 42, wherein the recipient subject is suffering from a disease and is in need of treatment, and wherein the exogenous material and/or the derivative thereof treats the disease.
44. The method of alternative 42 or 43, wherein administration of the platelet to the recipient subject induces immune protection against a virus, bacteria, or protozoan in the recipient subject.
45. The method of any one of alternatives 42-44, wherein the platelet comprising the exogenous material and/or the derivative thereof is administered to the recipient subject parenterally, intramuscularly, intraperitoneally, intravenously, subcutaneously, trans-hepatically, intra-vascularly, intra-hepatically, intra-portally, intra-splenically, or intradermally, optionally by ultrasound-guided injection.
46. The method of any one of alternatives 29-45, wherein the platelet and/or megakaryocyte is obtained or derived from a donor subject, optionally wherein the platelet and/or megakaryocyte is obtained or derived from pluripotent stem cells obtained or derived from the donor subject.
47. The method of alternative 46, wherein the platelet and/or megakaryocyte is obtained or derived from pluripotent stem cells obtained or derived from the donor subject according to a method comprising:
a) differentiating the pluripotent stem cells obtained or derived from the donor subject to hemogenic endoderm (HE);
b) differentiating the HE to CD34+ cells; and
c) differentiating the CD34+ cells to the megakaryocyte;
wherein the megakaryocyte produces the platelet.
48. The method of alternative 46 or 47, wherein the donor subject has been provided with a megakaryocyte stimulator.
49. The method of alternative 48, wherein the megakaryocyte stimulator is thrombopoietin, romiplostim, eltrombopag, avatrombopag, lusutrombopag, or any combination thereof.
50. The method of any one of alternatives 46-49, wherein the recipient subject or donor subject, or both, are mammals, optionally wherein the recipient subject and the donor subject are the same individual.
51. The method of alternative 50, wherein the recipient subject or donor subject, or both, are human.
52. The method of any one of alternatives 29-51, wherein contacting the platelet with the recipient cell(s) is allogeneic or autologous.
53. A platelet prepared by the method of any one of alternatives 1-28.
54. The platelet of alternative 53 for use in inducing immune protection against a virus, bacteria, or protozoan in a subject.
55. The platelet of alternative 54 for use in editing a gene in a recipient cell.
56. The platelet for use of alternative 55, wherein editing the gene in the recipient cell induces a desirable phenotype.
57. The platelet for use of alternative 55 or 56, wherein editing the gene in the recipient cell treats a disease in the recipient cell.
58. The platelet for use of alternative 57, wherein the disease is tyrosinemia type 1 and the edited gene is the Fah gene.
59. The platelet for use of alternative 57, wherein the disease is ornithine transcarbamylase deficiency and the edited gene is the OTC gene.
60. The platelet for use of alternative 57, wherein the disease is citrullinemia type 1 (CTLN1) and the edited gene is the argininosuccinate synthetase (ASS1) gene.
61. The platelet for use of alternative 57, wherein the disease is adult-onset type II citrullinemia (CTLN2) and the edited gene is the citrin (solute carrier family 25, member 13; SLC25A13) gene.
62. The platelet for use of any one of alternatives 55-61, wherein the gene in the recipient cell is edited in vitro, ex vivo, or in vivo.
63. The platelet for use of any one of alternatives 55-62, wherein the platelet is administered to a recipient subject.
64. The platelet for use of alternative 63, wherein the recipient subject is a mammal.
65. The platelet for use of alternative 64, wherein the recipient subject is a human.
66. The platelet for use of any one of alternatives 63-65, wherein the platelet is obtained or derived from the recipient subject, optionally wherein the platelet is obtained or derived from a megakaryocyte obtained or derived from the recipient subject, optionally wherein the platelet and/or megakaryocyte is obtained or derived from pluripotent stem cells obtained or derived from the recipient subject.
67. The platelet for use of alternative 66, wherein the platelet and/or megakaryocyte is obtained or derived from pluripotent stem cells obtained or derived from the recipient subject according to a method comprising:
a) differentiating the pluripotent stem cells obtained or derived from the recipient subject to hemogenic endoderm (HE);
b) differentiating the HE to CD34+ cells; and
c) differentiating the CD34+ cells to the megakaryocyte;
wherein the megakaryocyte produces the platelet.
68. A pharmaceutical composition comprising an effective amount of a platelet made by the method of any one of alternatives 1-28 and a pharmaceutically acceptable excipient, diluent, and/or carrier.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1 depicts an embodiment of a schematic overview of modified platelet-based content transfer.

FIG. 2A-D depict embodiments of platelet-mediated GFP mRNA delivery in vitro. GFP mRNA was delivered to HUVECs via platelets. Fluorescent imaging and flow cytometry were performed 24 hours after platelet delivery (FIG. 2A). Flow cytometry shows GFP signal in non-transfected platelets and GFP mRNA transfected platelets (FIG. 2B). Quantification of the percentage of GFP positive HUVECs at 24 or 48 hours after platelet delivery (FIG. 2C). GFP mRNA was delivered to HepG2 cells via platelets. Fluorescent imaging and flow cytometry were performed 24 hours after platelet delivery (FIG. 2D).

FIG. 3A-B depict embodiments of platelet-mediated GFP mRNA delivery in vitro. GFP mRNA were delivered to HUVECs via platelets (FIG. 3A). Fluorescent imaging and flow cytometry for FIG. 3A were performed 48 hours after platelet delivery. Images showing HUVECs expressing GFP proteins by Lipofectamine MessengerMAX control or the platelet delivery system at the time of platelet delivery (0 hour) or after 48 hours following delivery (FIG. 3B).

FIG. 4A-B depict embodiments of MEG-01 megakaryocytes infected with pLV-GFP. Flow cytometry was performed on the MEG-01-GFP generated platelets (FIG. 4A). MEG-01-GFP platelets were co-cultured with activated HUVECs. Images were taken 24 or 48 hours after co-culture (FIG. 4B).

FIG. 5A-C depict embodiments of platelet-mediated mCherry mRNA delivery in vitro to liver organoids. mCherry mRNA was transfected by Lipofectamine MessengerMAX into day 20 human liver organoids (FIG. 5A). mCherry mRNA was delivered by platelets into day 20 human liver organoids (FIG. 5B). Quantification of the percentage of mCherry positive human liver organoids after 24 hours (FIG. 5C).

FIG. 6A-C depict embodiments of co-culture of GFP-expressing platelets with mCherry-HUVECs. MEG-01-GFP megakaryocyte-derived platelets were co-cultured with activated mCherry-HUVECs. Z-stack images were taken by confocal microscopy at 24 hours following co-culture (FIG. 6A). The HUVECs were transfected with GFP mRNA using a Lipofectamine MessengerMAX control, GFP platelets, or GFP platelets with 80 μM dynasore. Images were taken after 24 hours following mRNA delivery (FIG. 6B). Scanning electron microscope images were taken 5 minutes after co-culture of activated HUVECs and platelets. Magnification: 2200× (left); 5000× (right) (FIG. 6C).

FIG. 7A-C depict embodiments of in vivo platelet-based mRNA delivery. Dose dependent in vivo luciferase expression quantitated as radiance by IVIS Spectrum CT at 24 and 48 hours post tail vein injection (FIG. 7A). Quantification of luciferase intensity at 24 and 48 hours post tail vein injection (FIG. 7B). Hematoxylin/cosin staining of liver and lung tissue at 24 hours post tail vein injection (FIG. 7C).

FIG. 8A-C depict embodiments of human platelet-based in vivo gene delivery to mouse cells. Ex vivo luminescent imaging of major organs excised 24 hours following platelet delivery using a sham control or platelets loaded with luciferase mRNA (200 million platelets+20 μg mRNA) in FRG or NSG mice (FIG. 8A). Luminescence is expressed in radiance (p/scc/cm²/sr). Histology (H&E) stain of major organs from FRG mice 24 hours post tail vein injection of 200 million (200M) platelets loaded with 20 μg of luciferase mRNA (FIG. 8B). FIG. 8C depicts another exemplary ex vivo luminescent imaging of major organs excised 24hours following platelet delivery with intrasplenic injection of platelets loaded with luciferase mRNA (200 million platelets+20 μg mRNA) in FRG mice, indicating hepatocyte targeted mRNA delivery with platelet infusion.

FIG. 9A-B depict embodiments of human platelet-based in vitro gene delivery to human HEK293T cells. pCAG-Cre-IRES-GFP transfection using 10 million (10M) platelets loaded with 1 μg plasmid by Lipofectamine 3000 in 293T cells, 24 hours post transfection (FIG. 9A). Sham pCAG-Cre-IRES-GFP transfection of 1 μg plasmid by Lipofectamine 3000 in 293T cells, 24 hours post transfection (FIG. 9B).

FIG. 10A-D depict embodiments of human platelet-based gene delivery to human cells. mCherry mRNA transfection using human platelets loaded by MessengerMAX in HepG2 cells (FIG. 10A), endothelial cells derived from human induced pluripotent stem cells (FIG. 10B), human iPSC lines (clone: 1383D6) (FIG. 10C), and liver organoid derived from iPSCs (FIG. 10D).

FIG. 11A-B depict embodiments of mouse platelet-based gene delivery to human cells. mCherry mRNA transfection using purified mouse platelet (PLT) loaded by MessengerMAX in human iPSC lines (clone: 1383D6), 48 hours post transfection (FIG. 11A). Flow cytometric assessment of mRNA transfected iPSCs for various platelet and mRNA amounts (FIG. 11B). The histograms were generated to show cells with mCherry fluorescence in comparison to the unstained control.

FIG. 12A-B depict embodiments of platelet surface modification to enhance cell type sensitivity. Senescent platelets lose sialic acid leading to exposure of penultimate surface galactose. Desialylated platelets more efficient bind to the hepatic Ashwell-Morell Receptor (AMR) on hepatocytes (FIG. 12A). Flow-cytometric analysis of lectin based galactose and sialic acid quantification suggest chilled platelets has more desialylated platelets (FIG. 12B).

FIG. 13A-D depicts embodiments for an exemplary approach for knocking out genes with indels using CRISPR/Cas9-mediated gene editing, such as for treating tyrosinemia type 1, which is caused by dysfunction of fumarylacetoacetate hydrolase (Fah). FIG. 13A depicts an exemplary schematic for correcting Fah function in a tyrosinemia type 1 model where a neomycin resistance cassette has been inserted within exon 5 of Fah. Platelets are loaded with assembled ribonucleoprotein (RNP) complex of Cas9 and a gRNA targeting the neomycin resistance cassette, and used to transfer the RNP complex to affected cells. To assess transfer efficiency, the tracrRNA of the RNP complex is conjugated to ATTO 550. FIG. 13B depicts the population of platelets that have been loaded with the RNP complex as detected by ATTO 550 signal. FIG. 13C depicts the presence of ATTO 550-labeled RNP complex in 1383D6 iPSCs contacted with RNP loaded platelets after 24 hours of incubation. FIG. 13D depicts the percentage of iPSCs positive for ATTO 550-labeled RNP complex proportional to the amount of RNP complex used.

FIG. 13E depicts an embodiment for an exemplary approach for introduction of point mutations using CRISPR/Cas9-mediated gene editing, such as for treating ornithine transcarbamylase (OTC) deficiency.

FIG. 14A-B depict embodiments of modifications of platelet targeting by ligand anchoring. Extracellular ligands on the surface of platelets allows for binding to proteins such as wheat germ agglutinin (WGA). Streptavidin complexes bound to biotin-conjugated WGA and biotin-conjugated ligands permit robust attachment of desired ligands to platelet surfaces (FIG. 14A). Genetic engineering of megakaryocyte with desired targeting ligands fused to transmembrane proteins permit the formation of platelets displaying the desired ligands (FIG. 14B).

FIG. 15A-B depict embodiments of identifying possible anchoring proteins. Exemplary constructs used to screen or identify cell surface targeting ligand proteins can be delivered to megakaryocytes, such as by viral transduction (FIG. 15A). Exemplary constructs such as those shown in FIG. 15A, testing CD41, CD42, and CD61, were transduced to 293T cells. Live cells were stained with a PE anti-HA antibody and also express eGFP by the construct. Flow cytometry gated for eGFP and detecting PE fluorescence shows that the C-terminal CD61-HA fusion construct results in anti-HA fluorescence on the cell surface (FIG. 15B).

FIG. 16 depicts an embodiment of protein translation efficiency of cells contacted with platelets loaded with mRNA encoding for fluorescent protein for different cell types. Conditions labeled with a “-” denote ones that have not been assessed.

FIG. 17A-C depict embodiments of inhibiting platelet-based delivery with an actin polymerization inhibitor (cytochalasin B). FIG. 17A shows representative fluorescent images of iPSCs expressing mCherry delivered by platelets with or without treatment with 100 nM or 350 nM cytochalasin B. FIG. 17B shows relative MFI and percentage of mCherry-positive cells in platelet-delivered iPSCs treated with cytochalasin B compared to control without cytochalasin B. FIG. 17C shows MFI for green fluorescence indicating dead cells in platelet-delivered iPSCs with or without treatment with cytochalasin B.

FIG. 18 depicts an embodiment of tracking platelet-based delivery using PKH67 staining, which generally labels cell membranes. Platelets loaded with mCherry mRNA were stained with PKH67, and then contacted with iPSCs. Double labeled PKH67+/mCherry+ iPSCs was dose-dependent on the amount of platelets added, suggesting that platelet transfer can be mediated by endocytosis.

FIG. 19A-C depict embodiments of platelet-mediated transfer of mitochondria. FIG. 19A shows relative quantification of human mitochondrial DNA (mtDNA) amplified from mouse embryonic fibroblasts (MEFs) with or without platelet contact, compared to human platelet control. FIG. 19B shows the quantification cycle of human mtDNA amplification from MEFs with or without platelet contact, compared to human platelet control. FIG. 19C shows the MFI of MEFs with or without platelet contact and human platelet control stained with CD41, which labels platelets.

FIG. 20A-D depict embodiments of megakaryocyte and platelet differentiation from CD34+ cells differentiated from iPSCs or derived from umbilical cord blood (UCB). FIG. 20A shows an exemplary protocol for differentiating iPSCs to megakaryocytes and platelets. FIG. 20B shows representative light microscope images of cells undergoing the exemplary differentiation protocol of FIG. 20A. FIG. 20C shows flow cytometric plots detecting populations of CD41 positive cells (labeling megakaryocytes and platelets) differentiated from CD34+ cells from iPSCs or UCB. FIG. 20D shows CD41 staining quantification of megakaryocytes and platelets differentiated from CD34+ cell from iPSCs or UCB. Four different batches of iPSC-differentiated cells were tested. FIG. 20E shows a population of CD41 positive/RNP (ATTO 550) positive cells in RNP loaded UCB derived platelets and 1383D6 iPSCs contacted with the RNP loaded UCB derived platelets.

### DETAILED DESCRIPTION

As disclosed herein, platelets, such as those isolated from human, are loaded with a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof and used as a novel delivery method with target cell specificity. These platelets can be used, for example, for gene editing, mRNA based gene therapy and vaccines, enzyme replacement therapies, or in vivo expression of therapeutic antibodies.

Platelets derive from megakaryocytes and are a natural cell type that circulates throughout the body. They have excellent ability to carry nucleic acids and proteins, and can be internalized by other cell type variants. As described herein, stabilized mRNA is successfully delivered to target cells using human platelets. This delivery method displays several advantages, including great biocompatibility as well as high delivery and expression efficiency. Since the mRNA is only expressed transiently, this method is ideal to deliver, for example, gene editing enzymes such as recombinases, CRISPR nucleases (e.g. Cas9), or base editors to perform a “hit-and-run” gene correction strategy. The whole procedure is robust, fast to perform, and scalable at low costs. In some embodiments, the platelets used can be derived from a subject, enabling autologous therapy. Therefore, the disclosure provided herein serves as a useful platform towards mRNA-based gene therapy, vaccine delivery, and protein replacement therapy.

Currently, in vivo gene delivery systems are viral-based or nanoparticle-based and primarily use DNA. AAV based viral delivery systems are widely used since they do not appear to be harmful to humans and are thought not to integrate into the genome. Nevertheless, a study in dogs hints towards a potential cancerogenic risk using AAVs as delivery vectors (Kaiser J., 2020). Moreover, AAVs are commonly occurring viruses and therefore about 70% of adults already carry antibodies against them, making them ineffectual. Ways to reduce the pre-existing immune response to AAV remains a significant area of inquiry in this field. On the other hand, nanoparticle-based methods typically lack tropism. Some of the nanoparticle material can persist in the human body for an extended period of time and lead to unpredictable reactions. Therefore, researchers have focused heavily on finding true biomimetic materials. In contrast, platelets are a natural cell type that is abundantly produced in the human body. They are easy to obtain from blood samples and in an autologous platelet-based delivery system, there is no concern regarding adverse immune reaction. Furthermore, while platelets have no nucleus, they often carry nucleic acids, proteins, or organelles derived from the originating megakaryocytes, suggesting its ability to contain cargo. Inside of the body, platelets naturally aggregate around a wounded area. However, as described herein, when there is no injury in a murine model, platelets preferentially deliver mRNA to lung and liver tissue. These features make platelets an excellent vehicle for gene delivery.

Virus based deliver methods costs days to assemble the virus in vitro and have high batch to batch variation. When injected into the body, the virus often has relatively low transfection efficiency and the effect needs days after delivery to be detectable. Conversely, as shown herein, platelet-based mRNA delivery and transgene expression needs only about 2 hours. The procedure of modifying and delivering the platelets do not require excessive processes and can be easily scaled up. Furthermore, unlike DNA, which needs to be transported to the nucleus to be functional, mRNA can be translated into protein as soon as it gets into the cell cytoplasm. Taking advantage of this, expression signal can be detected within the 2-hour post-delivery timeframe. In the murine model disclosed herein, this platelet system showed very high delivery efficiency. In addition, the stability of the in vitro transcribed mRNA used herein can be increased by using 5′ capping and poly(A) tailing. The platelet-based delivery methods disclosed herein have great promise for use in clinically viable mRNA-based gene therapies and vaccines.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Definitions

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article unless the context indicates otherwise. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or clement or group of steps or elements. By “consisting of” is meant including, and limited to, whatever follows the phrase “consisting of.” Thus, the phrase “consisting of” indicates that the listed elements are required or mandatory, and that no other clements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and means a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated hercin.

The terms “function” and “functional” as used herein have their plain and ordinary meaning as understood in light of the specification and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” has its plain and ordinary meaning as understood in light of the specification and may refer to a cell not contained in a multi-cellular organism or tissue.

As used herein, “in vivo” is given its plain and ordinary meaning in light of the specification and refers to the performance of a method inside living organisms, usually animals, mammals, including humans, and plants, as opposed to a tissue extract or dead organism.

As used herein, “ex vivo” is given its plain and ordinary meaning in light of the specification and refers to the performance of a method outside a living organism with little alteration of natural conditions.

As used herein, “in vitro” is given its plain and ordinary meaning in light of the specification and refers to the performance of a method outside of biological conditions, e.g., in a petri dish or test tube.

The terms “nucleic acid” or “nucleic acid molecule” as used herein have their plain and ordinary meaning as understood in light of the specification and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain clements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thercof.

RNA is a nucleic acid polymer molecule that plays a wide range of functions in biological systems. Messenger RNA is responsible for the expression of proteins derived from the sequence information stored in genomic DNA. During transcription, a pre-mRNA transcript is processed (e.g. intron splicing, 5′ capping, polyadenylation) and exported from the nucleus as a mature mRNA, which is free-floating through the cytoplasm until bound to a ribosome for translation. Other RNA molecules, including but not limited to noncoding RNA (ncRNA), antisense RNA (asRNA), long noncoding RNA (IncRNA), microRNA (miRNA), Piwi-interacting RNA (piRNA), small interfering RNA (siRNA), or short hairpin RNA (shRNA), or combinations thereof, play large roles in gene regulation, typically through binding and subsequent degradation or inactivation of complementary mRNA and pathways such as the RNA-induced silencing complex (RISC). This knowledge had led to a rich toolset by which to engineer cells to transiently express (mRNA) or downregulate expression of proteins by transfecting synthesized or isolated RNA molecules. Similarly, transport of RNA molecules during inter-cellular transfer (e.g. through TNTs or microvesicles) imparts considerable effects in recipient cells.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthinc, xanthinc, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

The term “gene editing protein” as used herein as its plain and ordinary meaning as understood in light of the specification and refers to a protein that is able to modify nucleic acids such as genomic DNA found in living organism. In some embodiments, the gene editing protein is able to insert or delete a nucleic acid fragment from the target nucleic acid. In some embodiments, the nucleic acid fragment can have a size that is, is about, is at least, is at least about, is not more than, or is not more than about, 20, 40, 60, 80, 100, 120, 140, 160, 180, 200, 300, 400, 500, 1000, 2000, 3000, 4000, 5000, 6000, 7000, 8000, 9000, 10000, 15000, or 20000 nucleotides long, or any size within a range defined by any two of the aforementioned sizes, for example, 20 to 20000 nucleotides, 500 to 10000 nucleotides, 1000 to 5000 nucleotides, 20 to 8000 nucleotides, or 2000 to 20000 nucleotides. In other embodiments, the gene editing protein modifies a few nucleotides from the target nucleic acid, such as 1, 2, 3, 4, 5, 6, 7, 8, 9, or 10 nucleotides. In some embodiments, this modification can be an insertion, deletion, or substitution to another nucleotide. The insertion, deletion, or substitution can be performed through the cell's endogenous non-homologous end joining or homology directed repair mechanisms, optionally with a template nucleic acid with the desired modification. Introduction of a template nucleic acid using a gene editing protein may be used to induce expression of a downstream product, such as a protein or RNA, to impart some phenotype on the target cell. Another common alternative, either with the introduction of a template nucleic acid or the induction of frame shift mutations, is to knock out expression of a target gene. Some examples of gene editing proteins include but are not limited to zinc finger nucleases, TALENS, CRISPR nucleases (e.g. Streptococcus pyogenes Cas9, Streptococcus thermophilus Cas9, Staphylococcus aureus Cas9, Neisseria meningitidis Cas9, Francisella novicidia Cas12a or Cas12b, Prevotella sp. p5-125 Cas13a, Cas13b, Cas13c or Cas13d, Porphyromonas gulae Cas13a, Cas13b, Cas13c, or Cas13d, Riemerella anatipestifer Cas13a, Cas13b, Cas13c, or Cas13d), or base editors (e.g. cytosine base editors, BE1, BE2, BE3, BE4, HF1-BE3, SaBE4,BE4-Gam, SaBE4-Gam, adenine base editors, ABE7.9, ABE7.10, VQR-ABE, VRER-ABE, SaKKH-ABE, NG-ABE, ABEmax, xABE, or CRISPR variant such as dCas9), or any combination thereof.

The term “purity” of any given substance, compound, or material as used herein has its plain and ordinary meaning as understood in light of the specification and refers to the actual abundance of the substance, compound, or material relative to the expected abundance. For example, the substance, compound, or material is, is about, is at least, is at least about, is not more than, or is not more than about, 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and is intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” have their plain and ordinary meaning as understood in light of the specification and can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more (e.g. at least 1, 3, 5, 10) of the following: antioxidants, such as ascorbic acid, low molecular weight (less than about 10 residues) polypeptides, proteins, such is scrum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urca, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. In some embodiments, the administration is an intra-arterial, intravenous, intraportal, intra-articular, intradermal, peritoneal, intramedullary injections, intrathecal, direct intraventricular, or intraperitoneal injection. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

The terms “platelet” and “thrombocytes” as used herein has its plain and ordinary meaning as understood in light of the specification and refers to small (about 2-4 μm diameter) anucleated cells found in the blood of mammals. Platelets originate as cytoplasmic fragments originating from megakaryocytes, which largely reside in bone marrow. One major function of platelets are to induce hemostasis in response to a vascular injury, but platelets are also known to contribute to an immune response to clear potential pathogens associated with the injury. In order to induce hemostasis, platelets adhere to the disrupted vascular endothelial cells by attaching to exposed extracellular matrix proteins such as von Willebrand factor, collagen and laminin. Subsequently, platelet activation first occurs through binding of soluble adenosine diphosphate (ADP) released by endothelial cells, inducing calcium ion influx to the platelet cytoplasm, morphological changes, and degranulation to release more platelet activation and hemostatic compounds. This cascade induces more platelets to aggregate to the wound along with concurrent coagulation in a fibrin matrix potentiated by thrombin. Inhibitors of platelet formation, aggregation, or activation (“platelet inhibitor”) include, but are not limited to, prostaglandin I2, nitric oxide, cyclooxygenase inhibitors, aspirin, triflusal, ADP receptor inhibitors, cangrelor, clopidogrel, prasugrel, ticagrelor, ticlopidine, phosphodiesterase inhibitors, cilostazol, protease-activated receptor-1 antagonists, vorapaxar, glycoprotein IIB/IIIA inhibitors, abciximab, eptifibatide, tirofiban, adenosine reuptake inhibotrs, dipyridamole, thromboxane inhibitors, terutroban, dynamin inhibitors, dynasore, MiTMAB, OcTMAB, Mdivi 1, P110, dynamin inhibitory peptide, dynole 34-2, MB 0223, endocytosis inhibitors, chlorpromazine, genistein, beta-cyclodextrin, amiloride hydrochloride, dyngo 4a, filipin, nystatin, monensin, chloroquine, wortmannin, Pitstop 2, or casin, or any combination thereof. Platelets can be activated (“platelet activator”) using compounds including, but not limited to, thrombin, ADP, calcium, thromboxane A2 (TXA2), platelet activating factor (PAF), cathepsin G, von Willebrand factor, collagen, fibrinogen, or laminin, or any combination thereof. Platelet production can be induced in a subject with the use of a megakaryocyte stimulator, including, but not limited to, thrombopoietin, romiplostim, eltrombopag, avatrombopag, lusutrombopag, or any combination thereof.

The term “inter-cellular transfer” as used herein has its plain and ordinary meaning as understood in light of the specification and refers to the transportation of material, such as biological material, from one cell to another. This material can be native (normally produced by the cell) or non-native (introduced). While the plasma membrane typically acts as a barrier, mechanisms exist for cells to exchange material including but not limited to fluids, salts, nutrients, sugars, small molecule compounds, lipids, organelles, mitochondria, endosomes, vesicles, proteins, polypeptides, peptides, antibodies, nucleic acids, DNA, or RNA, or any combination thereof. RNA may include mRNA, miRNA, siRNA, shRNA, or other types of RNA disclosed herein or known in the art and may result in expression of proteins (which may be native or non-native) or downregulation of gene expression through endogenous silencing pathways. Without being limited by any mechanism of action, inter-cellular transfer may occur through tunneling nanotubes (TNTs) or cytonemes, which are long actin-containing membrane protrusions that connect two or more cells and facilitate transport. As disclosed herein, these TNTs may be selective for a certain category of biological material (e.g. by size, polarity, charge, stability, etc) and even for certain species of one category (e.g. one RNA is transferred more efficiently than another). Pro-inflammatory stimuli, such as lipopolysaccharide (LPS) or IFN-γ, reduces TNT formation. Another method of inter-cellular transfer is through microvesicles and exosomes. These small membrane-bound vesicles have been shown to be able to carry RNA such as mRNA and miRNA. As shown herein, these two types of inter-cellular transfer can be differentiated by testing conditioned media or through a transwell assay, which will permit transfer of free floating microvesicles and exosomes, but not allow direct cell-to-cell contact, which is necessary for TNTs.

The term “tyrosinemia type 1” as used here has its plain and ordinary meaning as understood in light of the specification and refers to the autosomal recessive disorder caused by mutations in the fumarylacetoacetate hydrolase (Fah) enzyme. The Fah enzyme is expressed mainly in the liver and kidneys and catalyzes the degradation of fumarylacetoacetate to fumarate and acetoacetate. Loss of function of the Fah enzyme results in accumulation of fumarylacetoacetate and upstream compounds, as well as converted to succinylacetone. This accumulation may result in severe liver and renal toxicity as well as neuropathies.

The term “ornithine transcarbamylase deficiency” as used herein has its plain and ordinary meaning as understood in light of the specification and refers to the X-linked recessive disorder caused by mutation in the ornithine transcarbamylase (OTC) enzyme. The OTC enzyme is involved in the urea cycle within the liver and catalyzes ornithine and carbamoyl phosphate to citrulline. Loss of function of the OTC enzyme results in hyperammonemia, which may potentially lead to seizures, other neurological issues, and death.

The term “citrullinemia type 1 (CTLN1)” as used herein has its plain and ordinary meaning as understood in light of the specification and refers to the autosomal recessive disorder of metabolism caused by a deficiency of argininosuccinate synthetase (ASS1), which encodes a cytosolic urea cycle enzyme, argininosuccinate synthetase (ASS). ASS is expressed predominantly in the liver and kidney, where it plays a crucial role in ammonia detoxification. Loss of function of ASS results in urea cycle disorders characterized by hyperammonemia, which in severe forms can lead to profound metabolic disturbances and neurocognitive impairments.

The term “adult-onset type II citrullinemia (CTLN2)” as used herein has its plain and ordinary meaning as understood in light of the specification and refers to an adult-onset autosomal recessive disease caused by mutations in SLC25A13, which encodes a liver-specific aspartate/glutamate transporter called citrin, which is localized in the inner membrane of mitochondria. Loss of function of citrin results in hyperammonemia accompanied by recurrent episodes of neuropsychiatric manifestations, including aberrant behavior, nocturnal delirium, disorientation, consciousness disturbances, convulsive seizures, and coma.

### Methods of Making Modified Platelets

Disclosed herein are methods of preparing a platelet comprising exogenous material and/or a derivative thereof. Generally, these methods comprise contacting the platelet with the exogenous material and/or a derivative thereof. However, the platelet with the exogenous material and/or a derivative thereof may also be prepared by contacting a megakaryocyte with the exogenous material and/or the derivative thereof, and allowing the megakaryocyte to produce platelets comprising the exogenous material and/or the derivative thereof. As platelets are produced through the direct fragmentation of megakaryocyte membrane and cytoplasm, the resultant platelets will contain the exogenous material and/or the derivative thereof introduced into the megakaryocyte. While the platelets may contain the same exogenous material introduced into the megakaryocyte, the platelets may also contain a derivative of the exogenous material introduced into the megakaryocyte. These derivatives of the exogenous material will typically be a result of cellular functions acting on the exogenous material and may include, in some non-limiting embodiments, modifications to the exogenous material (such as the addition or removal of a functional group or moiety, or a processed form of the exogenous material, or other products of enzymatic or chemical reactions), or completely different molecules (such as proteins expressed from an exogenous nucleic acid). Similarly, as platelets include many of the biological components found in the parent megakaryocyte, the introduction of an exogenous material directly into a platelet may result in the formation of a derivative of the exogenous material through processes within the platelet that occur as in the megakaryocyte (e.g. modification of the exogenous material, such as by enzymatic or chemical processes, or production of different molecules such as expression of proteins from nucleic acid).

Disclosed herein are methods of preparing a platelet comprising exogenous material and/or a derivative thereof. In some embodiments, the methods comprise a) contacting the platelet with the exogenous material and/or the derivative thereof, or b) contacting a megakaryocyte with the exogenous material and/or the derivative thereof and allowing the megakaryocyte to produce the platelet comprising the exogenous material and/or the derivative thereof; thereby preparing the platelet comprising the exogenous material and/or the derivative thereof. In some embodiments, the exogenous material and/or the derivative thereof is biological material. In some embodiments, the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof. In some embodiments, the small molecule is a therapeutic small molecule. In some embodiments, the nucleic acid comprises DNA, or RNA, or both. In some embodiments, the RNA is or comprises mRNA, ncRNA, asRNA, lncRNA, miRNA, piRNA, siRNA, shRNA, gRNA, or any other type of RNA known in the art, or combinations thereof. In some embodiments, the nucleic acid encodes for one or more viral, bacterial, or protozoal proteins or a portion thereof. In some embodiments, the one or more viral, bacterial, or protozoal proteins or portion thereof is immunogenic, for example, in humans and other mammals. In some embodiments, the exogenous material and/or the derivative thereof comprises one or more viral, bacterial, or protozoal proteins. In some embodiments, the nucleic acid encodes for one or more gene editing proteins, for example, the gene editing proteins disclosed herein. In some embodiments, the exogenous material and/or the derivative thereof comprises one or more gene editing proteins, for example, the gene editing proteins disclosed herein. In some embodiments, the gene editing protein comprises a zinc finger nuclease, TALEN, nuclease, CRISPR nuclease, Cas9, or a base editor, or any combination thereof. In some embodiments, the exogenous material and/or the derivative thereof further comprises a gRNA or a nucleic acid encoding for the gRNA. This gRNA may be used to target a specific gene or genes for gene editing, particularly with the use of a CRISPR nuclease. In some embodiments, the exogenous material and/or the derivative thereof comprises one or more fluorescent proteins or one or more nucleic acids encoding for a fluorescent protein and successful transfer can be detected by measuring fluorescence of the recipient cells. In some embodiments, the exogenous material and/or the derivative thereof comprises one or more luminescent proteins or one or more nucleic acids encoding for a luminescent protein and successful transfer can be detected by measuring luminescence of the recipient cells. In some embodiments, contacting the platelet with the exogenous material and/or the derivative thereof or contacting the megakaryocyte with the exogenous material and/or the derivative thereof comprises transfection. In some embodiments, the transfection comprises electroporation, lipofection, or viral transduction. In some embodiments, following contacting of the exogenous material and/or the derivative thereof, the megakaryocytes are stimulated with a megakaryocyte stimulator, for example, the megakaryocyte stimulators described herein. In some embodiments, the megakaryocyte stimulator is thrombopoietin, romiplostim, eltrombopag, avatrombopag, lusutrombopag, or any combination thereof. In some embodiments, following contacting of the exogenous material and/or the derivative thereof, the platelets are activated using a platelet activator, for example, the platelet activators described herein. In some embodiments, the platelets are activated with thrombin, ADP, calcium, thromboxane A2 (TXA2), platelet activating factor (PAF), cathepsin G, von Willebrand factor, collagen, fibrinogen, or laminin, or any combination thereof.

In some embodiments, for option b) of the methods of producing platelets disclosed herein (where the method involves contacting the megakaryocyte with the exogenous material and/or the derivative thereof and allowing the megakaryocyte to produce the platelet comprising the exogenous material and/or the derivative thereof), i) the exogenous material and/or the derivative thereof comprised by the platelet is the exogenous material and/or the derivative thereof contacted with the megakaryocyte; or ii) the exogenous material and/or the derivative thereof comprised by the platelet is derived from the exogenous material and/or the derivative thereof contacted with the megakaryocyte. For example, the derivative of the exogenous material may be a protein translated from exogenous material that encodes for the protein (namely a nucleic acid). Accordingly, in some embodiments, the exogenous material and/or the derivative thereof comprised by the platelet comprises a protein and the exogenous material and/or the derivative thereof contacted with the megakaryocyte comprises a nucleic acid, where the protein is expressed from the nucleic acid. In some embodiments, the exogenous material and/or the derivative thereof comprised by the platelet comprises a first nucleic acid and the exogenous material and/or the derivative thereof contacted with the megakaryocyte comprises a second nucleic acid, where the first nucleic acid is the second nucleic acid or is derived from the second nucleic acid. In some embodiments, the exogenous material and/or the derivative thereof comprised by the platelet comprises a first protein and the exogenous material and/or the derivative thereof contacted with the megakaryocyte comprises a second protein, where the first protein is the second protein or a post-translationally modified variant of the second protein.

In some embodiments, the exogenous material and/or the derivative thereof comprises a nucleic acid, including but not limited to DNA, RNA, mRNA, ncRNA, asRNA, lncRNA, miRNA, piRNA, siRNA, shRNA, or gRNA. In some embodiments, the exogenous material and/or the derivative thereof comprising the nucleic acid is transfected. In some embodiments, the nucleic acid is transfected by electroporation, lipofection, or viral transduction. In some embodiments, the nucleic acid is transfected using the Lipofectamine MessengerMAX kit, Lipofectamine 3000 kit, or any other methodology known in the art. In some embodiments, the nucleic acid is contacted with platelets or megakaryocytes, or both, in an amount that is, is about, is at least, is at least about, is not more than, or is not more than about, 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 ng, or 1, 2, 3, 4, 5, 6, 7, 8, 9, 10, 11, 12, 13, 14, 15, 16, 17, 18, 19, 20, 30, 40, 50, 60, 70, 80, 90, or 100 μg of nucleic acid, or any amount within a range defined by any two of the aforementioned masses, for example, 1 ng to 100 μg, 100 ng to 50 μg, 1 to 1000 ng, 50 to 500 ng, 1 to 10 μg, 50 to 100 μg, or 5 to 20 μg of nucleic acid. In some embodiments, the platelets or megakaryocytes, or both are contacted at a number of cells that is, is about, is at least, is at least about, is not more than, or is not more than about, 1 million, 2 million, 3 million, 4 million, 5 million, 6 million, 7 million, 8 million, 9 million, 10 million, 20 million, 30 million, 40 million, 50 million, 60 million, 70 million, 80 million, 90 million, 100 million, 200 million, 300 million, 400 million, 500 million, 600 million, 700 million, 800 million, 900 million, or 1000 million platelets or megakaryocytes, or any number of platelets or megakaryocytes within a range defined by any two of the aforementioned number, for example, 1 million to 1000 million, 10 million to 500 million, 20 million to 50 million, or 500 million to 1000 million platelets or megakaryocytes. In some embodiments, the platelets or megakaryocytes are contacted at a ratio of 10, 20, 30, 40, 50, 60, 70, 80, 90, 100, 110, 120, 130, 140, 150, 160, 170, 180, 190, 200, 250, 300, 350, 400, 450, or 500 ng of nucleic acid per 1 million platelets or megakaryocytes, or any ratio within a range defined by any two of the aforementioned ratios, for example, 10 to 500 ng nucleic acid per 1 million platelets or megakaryocytes, 10 to 200 ng nucleic acid per 1 million platelets or megakaryocytes, 80 to 120 ng nucleic acid per 1 million platelets or megakaryocytes, or 200 to 500 ng nucleic acid per 1 million platelets or megakaryocytes. In some embodiments, following contacting of the nucleic acid, the platelets are activated using a platelet activator, for example, the platelet activators described herein. In some embodiments, the platelets are activated with thrombin, ADP, calcium, thromboxane A2 (TXA2), platelet activating factor (PAF), cathepsin G, von Willebrand factor, collagen, fibrinogen, or laminin, or any combination thereof.

A major advantage of the methods provided herein is that transfer of nucleic acids much larger than the limit seen in conventional methods is possible. For example, AAV vectors have a packaging capacity of about 4.7 kb of nucleic acid. This size limit has proven to be a hindrance for gene editing using the traditional S. pyogenes Cas9, which has a coding sequence length of 4.1 kb. Due to the much larger size of platelets compared to viral particles, there is virtually no limit in nucleic acid size that can be transferred. In some embodiments, the nucleic acids that can be used in the exogenous material and/or the derivative thereof is, is about, is at least, is at least about, is not more than, or is not more than about, 1, 10, 100, 1000, 10⁴, 10⁵, 10⁶, 10⁷, or 10⁸ bases in length, or any length within a range defined by any two of the aforementioned lengths, for example, 1 to 10⁸ bases, 1 to 10⁴ bases, 10⁴ to 10⁸ bases, or 100 to 10⁶ bases in length.

In some embodiments, the exogenous material and/or the derivative thereof comprises a nucleic acid. In some embodiments, the exogenous material and/or the derivative thereof comprises mRNA. In some embodiments, the mRNA is synthesized in vitro. In some embodiments, the mRNA comprises a functional 7-methylguanosine 5′ cap, or a poly (A) 3′ tail, or both. In some embodiments, the mRNA comprises 5-methoxyuridine (5-moU). In some embodiments, the mRNA comprises 5-methoxyuridine partially or fully substituting for uridine. In some embodiments, the mRNA (or any other nucleic acid) is purified.

In some embodiments, the exogenous material and/or the derivative thereof comprises a peptide or protein. In some embodiments, the peptide or protein is an antibody. In some embodiments, the exogenous material and/or the derivative thereof comprising the peptide or protein is transfected. In some embodiments, the peptide or protein is transfected by electroporation, lipofection, or with the use of protein transduction domains (including but not limited to the herpes simplex virus VP22 or HIV TAT protein transduction motif). In some embodiments, the peptide or protein is transfected using the Chariot Protein Delivery Reagent (ActiveMotif), Pierce Protein Transfection Reagent (Thermo Fisher), or any other methodology known in the art. In some embodiments, the peptide or protein is contacted with platelets or megakaryocytes, or both, in an amount that is, is about, is at least, is at least about, is not more than, or is not more than about, 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 ng, or 1, 2, 3, 4, 5, 6, 7, 8, 9, 10, 11, 12, 13, 14, 15, 16, 17, 18, 19, 20, 30, 40, 50, 60, 70, 80, 90, or 100 μg of peptide or protein, or any amount within a range defined by any two of the aforementioned masses, for example, 1 ng to 100 μg, 100 ng to 50 μg, 1 to 1000 ng, 50 to 500 ng, 1 to 10 μg, 50 to 100 μg, or 5 to 20 μg of peptide or protein. In some embodiments, the platelets or megakaryocytes, or both are contacted at a number of cells that is, is about, is at least, is at least about, is not more than, or is not more than about, 1 million, 2 million, 3 million, 4 million, 5 million, 6 million, 7 million, 8 million, 9 million, 10 million, 20 million, 30 million, 40 million, 50 million, 60 million, 70 million, 80 million, 90 million, 100 million, 200 million, 300 million, 400 million, 500 million, 600 million, 700 million, 800 million, 900 million, or 1000 million platelets or megakaryocytes, or any number of platelets or megakaryocytes within a range defined by any two of the aforementioned number, for example, 1 million to 1000 million, 10 million to 500 million, 20 million to 50 million, or 500 million to 1000 million platelets or megakaryocytes. In some embodiments, the platelets or megakaryocytes are contacted at a ratio of 10, 20, 30, 40, 50, 60, 70, 80, 90, 100, 110, 120, 130, 140, 150, 160, 170, 180, 190, 200, 250, 300, 350, 400, 450, or 500 ng of peptide or protein per 1 million platelets or megakaryocytes, or any ratio within a range defined by any two of the aforementioned ratios, for example, 10 to 500 ng peptide or protein per 1 million platelets or megakaryocytes, 10 to 200 ng peptide or protein per 1 million platelets or megakaryocytes, 80 to 120 ng peptide or protein per 1 million platelets or megakaryocytes, or 200 to 500 ng peptide or protein per 1 million platelets or megakaryocytes. In some embodiments, following contacting of the peptide or protein, the platelets are activated using a platelet activator, for example, the platelet activators described herein. In some embodiments, the platelets are activated with thrombin, ADP, calcium, thromboxane A2 (TXA2), platelet activating factor (PAF), cathepsin G, von Willebrand factor, collagen, fibrinogen, or laminin, or any combination thereof.

In some embodiments, the exogenous material and/or the derivative thereof comprises a small molecule. In some embodiments, the small molecule is a therapeutic small molecule. For example, the small molecule may be effective against a disease such as cancer. In some embodiments, the exogenous material and/or the derivative thereof comprising the small molecule is allowed to diffuse into the platelet or megakaryocyte. Alternatively, the small molecule can be transported with lipofection or other nanoparticle structures if the small molecule is membrane-impermeable. In some embodiments, the small molecule is contacted with platelets or megakaryocytes, or both, in an amount that is, is about, is at least, is at least about, is not more than, or is not more than about, 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 ng, or 1, 2, 3, 4, 5, 6, 7, 8, 9, 10, 11, 12, 13, 14, 15, 16, 17, 18, 19, 20, 30, 40, 50, 60, 70, 80, 90, or 100 μg of small molecule, or any amount within a range defined by any two of the aforementioned masses, for example, 1 ng to 100 μg, 100 ng to 50 μg, 1 to 1000 ng, 50 to 500 ng, 1 to 10 μg, 50 to 100 μg, or 5 to 20 μg of small molecule. In some embodiments, the platelets or megakaryocytes, or both are contacted at a number of cells that is, is about, is at least, is at least about, is not more than, or is not more than about, 1 million, 2 million, 3 million, 4 million, 5 million, 6 million, 7 million, 8 million, 9 million, 10 million, 20 million, 30 million, 40 million, 50 million, 60 million, 70 million, 80 million, 90 million, 100 million, 200 million, 300 million, 400 million, 500 million, 600 million, 700 million, 800 million, 900 million, or 1000 million platelets or megakaryocytes, or any number of platelets or megakaryocytes within a range defined by any two of the aforementioned number, for example, 1 million to 1000 million, 10 million to 500 million, 20 million to 50 million, or 500 million to 1000 million platelets or megakaryocytes. In some embodiments, the platelets or megakaryocytes are contacted at a ratio of 10, 20, 30, 40, 50, 60, 70, 80, 90, 100, 110, 120, 130, 140, 150, 160, 170, 180, 190, 200, 250, 300, 350, 400, 450, 500, 600, 700, 800, 900, or 1000 ng, or 1, 2, 3, 4, 5, 6, 7, 8, 9, 10, 20, 30, 40, 50, 60, 70, 80, 90, 100, 200, 300, 400, 500, 600, 700, 800, 900 or 1000 μg of small molecule per 1 million platelets or megakaryocytes, or any ratio within a range defined by any two of the aforementioned ratios, for example, 10 ng to 1000 μg small molecule per 1 million platelets or megakaryocytes, 10 to 1000 ng small molecule per 1 million platelets or megakaryocytes, 1 to 1000 μg small molecule per 1 million platelets or megakaryocytes, or 500 ng to 500 μg small molecule per 1 million platelets or megakaryocytes. In some embodiments, following contacting of the small molecule, the platelets are activated using a platelet activator, for example, the platelet activators described herein. In some embodiments, the platelets are activated with thrombin, ADP, calcium, thromboxane A2 (TXA2), platelet activating factor (PAF), cathepsin G, von Willebrand factor, collagen, fibrinogen, or laminin, or any combination thereof.

In some embodiments, platelets comprising exogenous material and/or the derivative thereof are isolated from modified megakaryocytes (e.g. megakaryocytes comprising exogenous material and/or the derivative thereof). In some embodiments, megakaryocytes are isolated from a donor subject. In some embodiments, the donor subject is a mammal. In some embodiments, the donor subject is a human. In some embodiments, the donor subject is treated with a megakaryocyte stimulator. In some embodiments, the megakaryocyte stimulator is thrombopoietin, romiplostim, eltrombopag, avatrombopag, lusutrombopag, or any combination thereof. In some embodiments, the donor subject is treated with a PBMC mobilizer. In some embodiments, the megakaryocytes are isolated from a blood sample of the donor subject. In some embodiments, the isolated megakaryocytes are contacted with the exogenous material and/or the derivative thereof. In some embodiments, the contacted megakaryocytes (e.g. megakaryocytes comprising exogenous material and/or the derivative thereof) are treated with a megakaryocyte stimulator, for example, the megakaryocyte stimulators disclosed herein. In some embodiments, the megakaryocyte stimulator is thrombopoietin, romiplostim, eltrombopag, avatrombopag, lusutrombopag, or any combination thereof. In some embodiments, platelets comprising the exogenous material and/or the derivative thereof are isolated from the contacted megakaryocytes.

As applied to any of the platelets comprising exogenous material and/or the derivative thereof disclosed herein, the platelet further comprises a cell surface modification that enhances selectively to a specific recipient cell type or types. In some embodiments, the cell surface modification comprises a modification to the surface glycans of the platelet. In some embodiments, the cell surface modification comprises desialylation of the surface glycans of the platelet. In some embodiments, the cell surface modification comprises an exogenous targeting ligand on or associated with the cell surface of the platelet. In some embodiments, the exogenous targeting ligand is selective for the specific recipient cell type or types. In the exogenous targeting ligand is biotinylated and is bound to a streptavidin complex, which is further found to a biotinylated platelet-specific ligand that is bound to the platelet. In some embodiments, the exogenous targeting ligand comprises a transmembrane component and an extracellular component. In some embodiments, the transmembrane component is a protein normally found in platelets. In some embodiments, the protein normally found in platelets comprises CD41, CD42a, CD42b, CD61, CD9, CD29, CD31, CD36, CD62P, CD63, CD107a, CD154, glycoprotein VI, integrin αIIbβ3, or any combination thereof. In some embodiments, the protein normally found in platelets comprises CD41, CD42a, CD42b, or CD61, which are the major markers for platelets. In some embodiments, the exogenous targeting ligand is expressed by the platelet and/or the megakaryocyte from which the platelet is derived.

### Methods of Using Modified Platelets

Disclosed herein are methods of delivering exogenous material and/or the derivative thereof to cells in vitro. In some embodiments, the platelets comprising exogenous material and/or the derivative thereof are contacted with recipient cells in vitro. In some embodiments, the recipient cells are mammalian cells. In some embodiments, the recipient cells are human cells. In some embodiments, the recipient cells are stem cells, induced pluripotent stem cells (iPSCs), human umbilical vein endothelial cells (HUVECs), HepG2, or organoids (e.g. organoids derived from stem cells), for example, liver organoids. In some embodiments, successful transfer of the exogenous material and/or the derivative thereof to the recipient cells is detected as early as a time that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 21, 22, 23, or 24 hours after contacting the recipient cells with the platelets comprising exogenous material and/or the derivative thereof. In some embodiments, the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof. In some embodiments, the nucleic acid comprises DNA, or RNA, or both. In some embodiments, the RNA comprises mRNA, miRNA, siRNA, shRNA, or any other type of RNA known in the art. In some embodiments, the nucleic acid encodes for one or more viral, bacterial, or protozoal proteins. In some embodiments, the exogenous material and/or the derivative thereof comprises one or more viral, bacterial, or protozoal proteins. In some embodiments, the one or more viral, bacterial, or protozoal protein is immunogenic, for example, in humans. In some embodiments, the nucleic acid encodes for one or more gene editing proteins, for example, the gene editing proteins disclosed herein. In some embodiments, the exogenous material and/or the derivative thereof comprises one or more gene editing proteins. In some embodiments, the gene editing protein comprises a zinc finger nuclease, TALEN, nuclease, CRISPR nuclease, Cas9, or a base editor, or any combination thereof. In some embodiments, the gene editing protein modifies the genome of the recipient cells. In some embodiments, the gene editing protein modifies the genome by homology directed repair or non-homologous end joining. In some embodiments, the modification of the genome of the recipient cells corrects an error in a gene. In some embodiments, the modification of the genome of the recipient cells treats a disease. In some embodiments, the modification of the genome comprises a modification to the fumarylacetoacetate hydrolase (Fah) gene. In some embodiments, the modification of the genome comprises a modification to the ornithine transcarbamylase (OTC) gene. In some embodiments, the modification of the genome comprises a modification to the argininosuccinate synthetase (ASS1) gene. In some embodiments, the modification of the genome comprises a modification to the citrin (solute carrier family 25, member 13; SLC25A13) gene. In some embodiments, the exogenous material and/or the derivative thereof comprises one or more fluorescent proteins or one or more nucleic acids encoding for a fluorescent protein and successful transfer can be detected by measuring fluorescence of the recipient cells. In some embodiments, the exogenous material and/or the derivative thereof comprises one or more luminescent proteins or one or more nucleic acids encoding for a luminescent protein and successful transfer can be detected by measuring luminescence of the recipient cells.

Also disclosed herein are methods of delivering exogenous material and/or the derivative thereof to a recipient subject ex vivo. The methods comprise contacting a platelet comprising exogenous material and/or the derivative thereof to donor cells isolated from a donor subject, and administering the contacted recipient cells to the recipient subject. The donor cells will exhibit a desired phenotype after contacting with the platelets, and subsequently, a desired effect will be experienced by the recipient subject. For example, the contacted donor cells are administered to the recipient subject to treat a disease. In some embodiments, the platelet comprising exogenous material and/or the derivative thereof is prepared by any one of the methods disclosed herein. In some embodiments, the donor subject and the recipient subject are the same individual such that the administration of the platelet comprising the exogenous material and/or the derivative thereof to the recipient subject is autologous. In some embodiments, the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof. In some embodiments, the nucleic acid comprises DNA, or RNA, or both. In some embodiments, the RNA comprises mRNA, miRNA, siRNA, shRNA, or any other type of RNA known in the art. In some embodiments, the nucleic acid encodes for one or more viral, bacterial, or protozoal proteins. In some embodiments, the exogenous material and/or the derivative thereof comprises one or more viral, bacterial, or protozoal proteins. In some embodiments, the one or more viral, bacterial, or protozoal protein is immunogenic, for example, in humans. In some embodiments, the nucleic acid encodes for one or more gene editing proteins, for example, the gene editing proteins disclosed herein. In some embodiments, the exogenous material and/or the derivative thereof comprises one or more gene editing proteins. In some embodiments, the gene editing protein comprises a zinc finger nuclease, TALEN, nuclease, CRISPR nuclease, Cas9, or a base editor, or any combination thereof. In some embodiments, the gene editing protein modifies the genome of the recipient subject or a plurality of cells of the recipient subject thereof. In some embodiments, the gene editing protein or exogenous material and/or the derivative thereof modifies the genome of the recipient subject or a plurality of cells of the recipient subject thereof by homology directed repair or non-homologous end joining. In some embodiments, the plurality of cells of the recipient subject is an organ or tissue. In some embodiments, the modification of the genome of the recipient subject or a plurality of cells of the recipient subject thereof treats a disease. In some embodiments, the disease is tyrosinemia type 1 and the modification to the genome comprises a modification to the fumarylacetoacetate hydrolase (Fah) gene. In some embodiments, the disease is ornithine transcarbamylase deficiency and the modification to the genome comprises a modification to the ornithine transcarbamylase (OTC) gene. In some embodiments, the disease is citrullinemia type 1 (CTLN1) and the modification to the genome comprises a modification to the argininosuccinate synthetase (ASS1). In some embodiments, the disease is adult-onset type II citrullinemia (CTLN2) and the modification to the genome comprises a modification to the citrin (solute carrier family 25, member 13; SLC25A13) gene.

Also disclosed herein are methods of delivering exogenous material and/or the derivative thereof to a recipient subject in vivo. The methods comprise administering a platelet comprising exogenous material and/or the derivative thereof to the recipient subject. In some embodiments, the platelet comprising exogenous material and/or the derivative thereof is prepared by any one of the methods disclosed herein. In some embodiments, the platelet comprising the exogenous material and/or the derivative thereof was obtained from the recipient subject such that the administration of the platelet comprising the exogenous material and/or the derivative thereof to the recipient subject is autologous. In some embodiments, the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof. In some embodiments, the nucleic acid comprises DNA, or RNA, or both. In some embodiments, the RNA comprises mRNA, miRNA, siRNA, shRNA, or any other type of RNA known in the art. In some embodiments, the nucleic acid encodes for one or more viral, bacterial, or protozoal proteins. In some embodiments, the exogenous material and/or the derivative thereof comprises one or more viral, bacterial, or protozoal proteins. In some embodiments, the one or more viral, bacterial, or protozoal protein is immunogenic, for example, in humans. In some embodiments, the nucleic acid encodes for one or more gene editing proteins, for example, the gene editing proteins disclosed herein. In some embodiments, the exogenous material and/or the derivative thereof comprises one or more gene editing proteins. In some embodiments, the gene editing protein comprises a zinc finger nuclease, TALEN, nuclease, CRISPR nuclease, Cas9, or a base editor, or any combination thereof. In some embodiments, the gene editing protein or exogenous material and/or the derivative thereof modifies the genome of the recipient subject or a plurality of cells of the recipient subject thereof. In some embodiments, the gene editing protein or exogenous material and/or the derivative thereof modifies the genome of the recipient subject or a plurality of cells of the recipient subject thereof by homology directed repair or non-homologous end joining. In some embodiments, the plurality of cells of the recipient subject is an organ or tissue. In some embodiments, the modification of the genome of the recipient subject or a plurality of cells of the recipient subject thereof treats a disease. In some embodiments, the disease is tyrosinemia type 1 and the modification to the genome comprises a modification to the fumarylacetoacetate hydrolase (Fah) gene. In some embodiments, the disease is ornithine transcarbamylase deficiency and the modification to the genome comprises a modification to the ornithine transcarbamylase (OTC) gene. In some embodiments, the disease is citrullinemia type 1 (CTLN1) and the modification to the genome comprises a modification to the argininosuccinate synthetase (ASS1). In some embodiments, the disease is adult-onset type II citrullinemia (CTLN2) and the modification to the genome comprises a modification to the citrin (solute carrier family 25, member 13; SLC25A13) gene.

Also disclosed herein are methods of delivering exogenous material and/or a derivative thereof to a recipient cell. In some embodiments, the methods comprise contacting a platelet comprising the exogenous material and/or the derivative thereof with the recipient cell, thereby delivering the exogenous material and/or the derivative thereof to the recipient cell. In some embodiments, the platelet is produced by any of the methods disclosed herein. In some embodiments, the platelet comprises a gene editing protein and the gene editing protein modifies the genome of the recipient cell. In some embodiments, the gene editing protein modifies the genome of a plurality of recipient cells. In some embodiments, the plurality of recipient cells is part of an organ or tissue. In some embodiments, the recipient cell(s) is/are characterized by a disease and the modification to the genome of the recipient cell(s) treats the disease. In some embodiments, the disease is tyrosinemia type 1 and the modification to the genome comprises a modification to the fumarylacetoacetate hydrolase (Fah) gene. In some embodiments, the disease is ornithine transcarbamylase deficiency and the modification to the genome comprises a modification to the ornithine transcarbamylase (OTC) gene. In some embodiments, the disease is citrullinemia type 1 (CTLN1) and the modification to the genome comprises a modification to the argininosuccinate synthetase (ASS1). In some embodiments, the disease is adult-onset type II citrullinemia (CTLN2) and the modification to the genome comprises a modification to the citrin (solute carrier family 25, member 13; SLC25A13) gene. In some embodiments, the contacting occurs in vitro. In some embodiments, the contacting occurs ex vivo. In some embodiments, said recipient cell(s) are obtained or derived from a recipient subject, and the methods further comprise administering the recipient cell(s) to the recipient subject following the step of contacting the platelet with the recipient cell(s). In some embodiments, the contacting occurs in vivo. In some embodiments, said recipient cell(s) is/are in a recipient subject, and said contacting comprises administering said platelet to said recipient subject. In some embodiments, the recipient subject is suffering from a disease and is in need of treatment, and wherein the exogenous material and/or the derivative thereof treats the disease. In some embodiments, administration of the platelet to the recipient subject induces immune protection against a virus, bacteria, or protozoan in the recipient subject. In some embodiments,

In some embodiments, the platelet comprising exogenous material and/or the derivative thereof is administered to the recipient subject parenterally, intramuscularly, intraperitoneally, intravenously, subcutaneously, trans-hepatically, intra-vascularly, intra-hepatically, intra-portally, intra-splenically, or intradermally. In some embodiments, the platelet comprising exogenous material and/or the derivative thereof is administered to the recipient subject by ultrasound-guided injection. In some embodiments, the platelets comprising exogenous material and/or the derivative thereof are administered to the recipient subject at a number of platelets that is, is about, is at least, is at least about, is not more than, or is not more than about, 1 million, 2 million, 3 million, 4 million, 5 million, 6 million, 7 million, 8 million, 9 million, 10 million, 20 million, 30 million, 40 million, 50 million, 60 million, 70 million, 80 million, 90 million, 100 million, 200 million, 300 million, 400 million, 500 million, 600 million, 700 million, 800 million, 900 million, or 1000 million platelets, or any number of platelets within a range defined by any two of the aforementioned number, for example, 1 million to 1000 million, 10 million to 500 million, 20 million to 50 million, or 500 million to 1000 million platelets. In some embodiments, the platelets comprising exogenous material and/or the derivative thereof are administered to the recipient subject in an isotonic solution. In some embodiments, the isotonic solution is 0.9% saline, Ringer's lactate solution, Ringer's acetate solution, or 5% dextrose solution, or any combination thereof.

In some embodiments, the platelet and/or megakaryocyte of any of the methods of use provided herein is obtained or derived from a donor subject. In some embodiments, the platelet and/or megakaryocyte is obtained or derived from pluripotent stem cells obtained or derived from the donor subject. In some embodiments, the donor subject is a mammal. In some embodiments, the donor subject is a human. In some embodiments, the platelet and/or megakaryocyte is obtained or derived from pluripotent stem cells obtained or derived from the donor subject according to a method comprising: a) differentiating the pluripotent stem cells obtained or derived from the donor subject to hemogenic endoderm (HE); b) differentiating the HE to CD34+ cells; and c) differentiating the CD34+ cells to the megakaryocyte (where in some embodiments, the differentiated megakaryocyte produces the platelet). Methods for generating hemogenic endoderm and subsequent CD34+ from pluripotent stem cells are generally known in the art, for example, in Ohta R et al. “Hemogenic Endothelium Differentiation from Human Pluripotent Stem Cells in A Feeder- and Xeno-free Defined Condition.” J. Vis. Exp. (2019); 148, e59823, which is hereby expressly incorporated by reference in its entirety. Methods for generating CD34+ cells to the megakaryocyte are generally known in the art, for example, using the HemaTox Megakaryocyte Kit (StemCell Technologies) or Perdomo et al. “Megakaryocyte Differentiation and Platelet Formation from Human Cord Blood-derived CD34+ Cells” J. Vis. Exp. (2017); 130, 56420, each of which is hereby expressly incorporated by reference in its entirety. In some embodiments, the platelets are isolated from the donor subject by platelet apheresis. In some embodiments, the donor subject is first provided with a megakaryocyte stimulator, for example, the megakaryocyte stimulators disclosed herein. In some embodiments, the megakaryocyte stimulator is thrombopoietin romiplostim, eltrombopag, avatrombopag, lusutrombopag, or any combination thereof. In some embodiments, the recipient subject is a mammal. In some embodiments, the recipient subject is a human. In some embodiments, both the donor subject or donor cell(s) and recipient subject or recipient cell(s) are human, and the administration of the platelet comprising exogenous material and/or the derivative thereof is allogeneic. In some embodiments, the donor subject and recipient subject is the same individual (or the donor cell(s) and the recipient cell(s) are from the same individual), and the administration of the platelet comprising exogenous material and/or the derivative thereof is autologous. In some embodiments, the platelet is obtained or derived from the recipient subject. In some embodiments, the platelet is obtained or derived from a megakaryocyte obtained or derived from the recipient subject. In some embodiments, the platelet and/or megakaryocyte is obtained or derived from pluripotent stem cells obtained or derived from the recipient subject. In some embodiments, the platelet and/or megakaryocyte is obtained or derived from pluripotent stem cells obtained or derived from the recipient subject according to a method comprising: a) differentiating the pluripotent stem cells obtained or derived from the recipient subject to hemogenic endoderm (HE); b) differentiating the HE to CD34+ cells; and c) differentiating the CD34+ cells to the megakaryocyte (where in some embodiments, the differentiated megakaryocyte produces the platelet). In some embodiments, the recipient subject has a disease, had a disease previously, or is at risk of contracting a disease. In some embodiments, the platelet comprising exogenous material and/or the derivative thereof is administered to the recipient subject as a therapeutic.

Disclosed herein is the platelet produced by any one of the methods disclosed herein. In some embodiments are platelet compositions comprising an effective amount of a platelet comprising exogenous material and/or the derivative thereof and one or more pharmaceutically acceptable excipient, diluent, or carriers. In some embodiments, the platelet comprising exogenous material and/or the derivative thereof is the platelet produced by any one of the methods disclosed herein. In some embodiments, the platelet compositions are pharmaceutical compositions.

Also disclosed herein is the use of a platelet comprising exogenous material and/or the derivative thereof in a subject as a therapeutic. In some embodiments, the platelet comprising exogenous material and/or the derivative thereof is the platelet produced by any one of the methods disclosed herein. In some embodiments, the platelet comprising exogenous material and/or the derivative thereof is used to induce immune protection against a virus, bacteria, or protozoan in a subject. In some embodiments, the platelet comprising exogenous material and/or the derivative thereof is used to edit a gene in a recipient cell or subject. In some embodiments, editing the gene in the recipient cell or subject induces a desirable phenotype. In some embodiments, editing the gene in the recipient cell or subject treats a disease in the recipient cell or subject. In some embodiments, the disease is tyrosinemia type 1 and the edited gene is the Fah gene. In some embodiments, the disease is ornithine transcarbamylase deficiency and the edited gene is the OTC gene. In some embodiments, the disease is citrullinemia type 1 (CTLN1) and the edited gene is the argininosuccinate synthetase (ASS1) gene. In some embodiments, the disease is adult-onset type II citrullinemia (CTLN2) and the edited gene is the citrin (solute carrier family 25, member 13; SLC25A13) gene. In some embodiments, the gene in the recipient cell is edited in vitro, ex vivo, or in vivo. In some embodiments, the desired therapeutic effect of the platelet comprising exogenous material and/or the derivative thereof is seen in the subject is seen in a time that is, is about, is at least, is at least about, is not more than, or is not more than about, 1, 2, 3, 4, 5, 6, 7, 8, 9, 10, 11, 12, 13, 14 days, 2, 3, 4, 5, 6, 7, 8 weeks, or 2, 3, 4, 5, 6, 7, 8, 9, 10, 11, or 12 months, or any time within a range defined by any two of the aforementioned times, for example, 1 to 14 days, 2 to 8 weeks, 2 to 12 months, or 1 day to 12 months, following administration of the platelet comprising exogenous material and/or the derivative thereof. In some embodiments, the subject is a mammal. In some embodiments, the subject is a human.

Also disclosed herein in some embodiments are pharmaceutical compositions that comprise, consist essentially of, or consist of an effective amount of a cell composition described herein and a pharmaceutically acceptable carrier, excipient, and/or carrier, or combinations thereof. The cell composition may be any one of the platelets and/or megakaryocytes described herein. The pharmaceutically acceptable carriers, excipients, and carriers may include those described herein or otherwise generally known in the art. They may be used for various purposes, such as maintaining isotonicity and pH of the pharmaceutical composition, improving viability of the cells, such as during the process of preparation, storage, cryopreservation, or administration, or improving efficacy of the desired outcome following administration. A pharmaceutical composition described herein is suitable for human and/or veterinary applications.

The cell compositions described herein (e.g. those including modified platelets and/or megakaryocytes) or pharmaceutical compositions thereof may be stored at cryogenic temperatures to maintain long-term viability and/or safety. Cryopreservation of biological cells is generally known in the art, and platelets can be reliably stored at −80° C. in a 5-6% DMSO solution for up to 2 years. However, at refrigerated, above freezing temperatures (below 16° C.), platelets exhibit significant degeneration in activity as soon as 24 hours, which is suspected to be attributed to their glycoprotein expression and morphological changes. Therefore, platelets are normally stored at room temperature (20-24° C.) for up to 5 days, after which bacterial contamination becomes a concern.

As the platelets described herein undergo processes to incorporate exogenous material and/or derivatives of the exogenous material, they must be freely manipulatable. Compositions comprising a RhoA inhibitor, a RAC inhibitor, a Cdc42 inhibitor, or combinations thereof have been shown to significantly improve the long-term storage of platelets at refrigerated temperatures (refer to PCT publication WO 2016/204809). Therefore, it is envisioned that compositions such as these may be added to isolated platelets to improve their viability during the methods described herein. Modified platelet preparations can then be stored at cryogenic temperatures for subsequent administration and other applications.

The disclosure is generally disclosed herein using affirmative language to describe the numerous embodiments. The disclosure also includes embodiments in which subject matter is excluded, in full or in part, such as substances or materials, method steps and conditions, protocols, or procedures.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Packaging of mRNA into Platelets

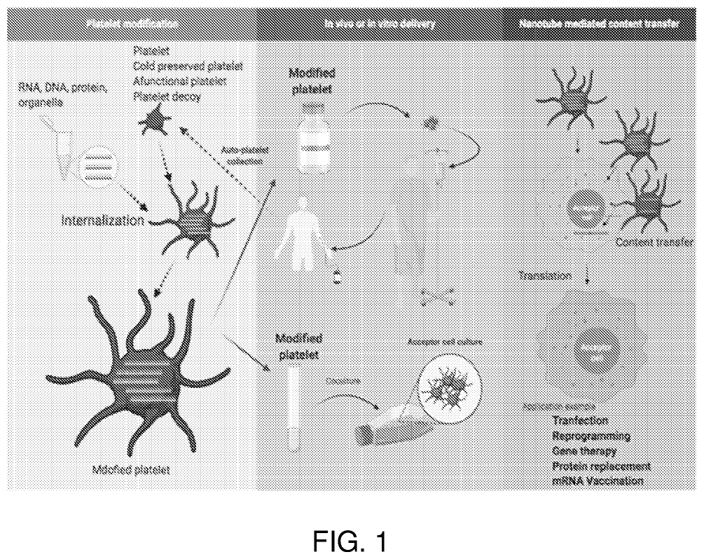

A schematic for platelet-based delivery is depicted in FIG. 1. mRNA was synthesized in vitro, for example, using the NEB HiScribe T7 ARCA mRNA kit (New England Biolabs), including necessary 7-methylguanosinc (m⁷G) 5′ capping and poly (A) 3′ tailing. Uridine can optionally be substituted to 5-methoxyuridine (5-moU) for reduced immunogenicity. After synthesis, mRNA is purified by standard methods.

The purified mRNA was transfected into isolated platelets, for example, using the Lipofectamine MessengerMAX kit (Thermo Fisher). Briefly, 8 μL of Lipofectamine was added to 400 μL of Opti-MEM I reduced serum media (Thermo Fisher) in a tube, mixed well, and incubated at room temperature for 5 minutes. In a separate tube, 5 μg of mRNA was added to 400 μL of Opti-MEM and mixed well. The contents of both tubes were combined, vortexed for 3 seconds, and incubated at room temperature for 15 minutes. Fresh human platelets were washed using Endothelial Cell Growth Basal Medium (EBM; Lonza). The platelets were centrifuged at 1800×g at room temperature for 10 minutes, and the supernatant was discarded. This wash step was repeated once. The platelet pellet was the resuspended in fresh EBM. 45 million pre-washed platelets were added to the combined mRNA/Lipofectamine/Opti-MEM transfection reagent in a silicone-coated tube and incubated at 37° C. for 1 hour.

### Example 2. In Vitro Delivery of Modified Platelets

Platelet-based mRNA delivery in HUVEC and HepG2 cell lines were tested. The cells were prepared 1 day before platelet delivery. On the day of transfection, the cells needed to reach 90-100% confluence. For a 24-well tissue culture plate, 1-5 μg of mRNA can be used for 3 wells divided equally. The mRNA/platelet transfection mixture was further prepared by centrifuging at 1800×g at room temperature for 10 minutes, washing with EBM, and finally resuspending in fresh 500 μL EBM. The growth medium was removed from recipient cells and the resuspended platelets were added to the cells. Thrombin (final concentration of 0.1 U/mL) was added to activate the platelets. The tissue culture plate was incubated at 37° C. for 1 hour, the EBM is discarded and changed for the appropriate culture medium for the recipient cells.

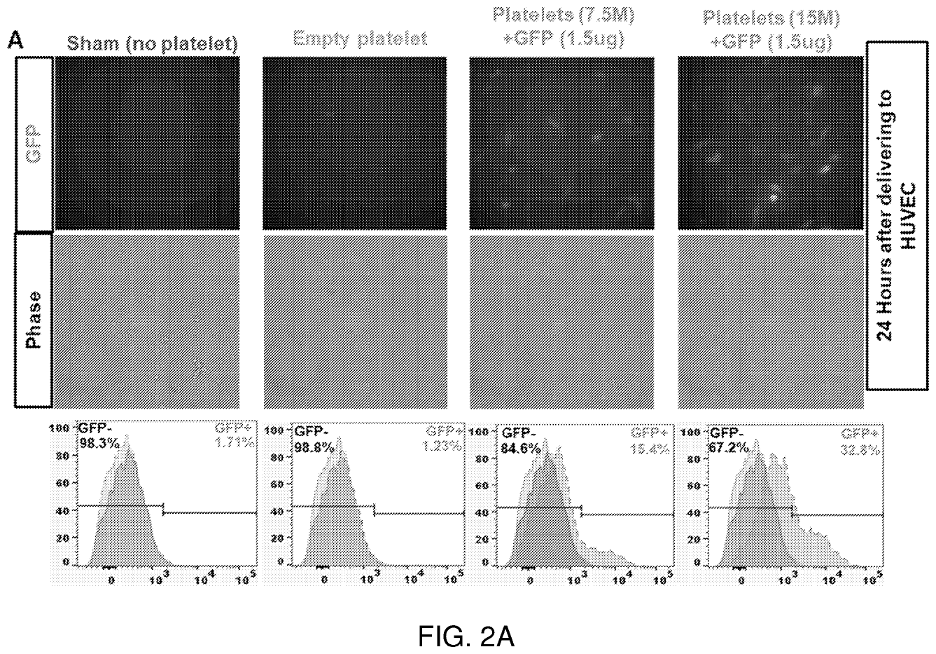

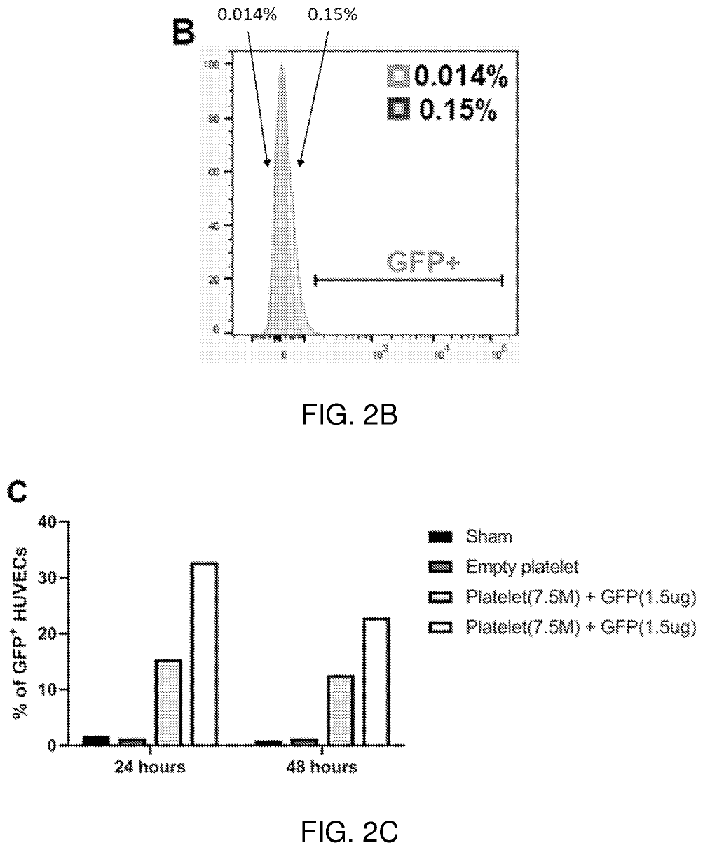

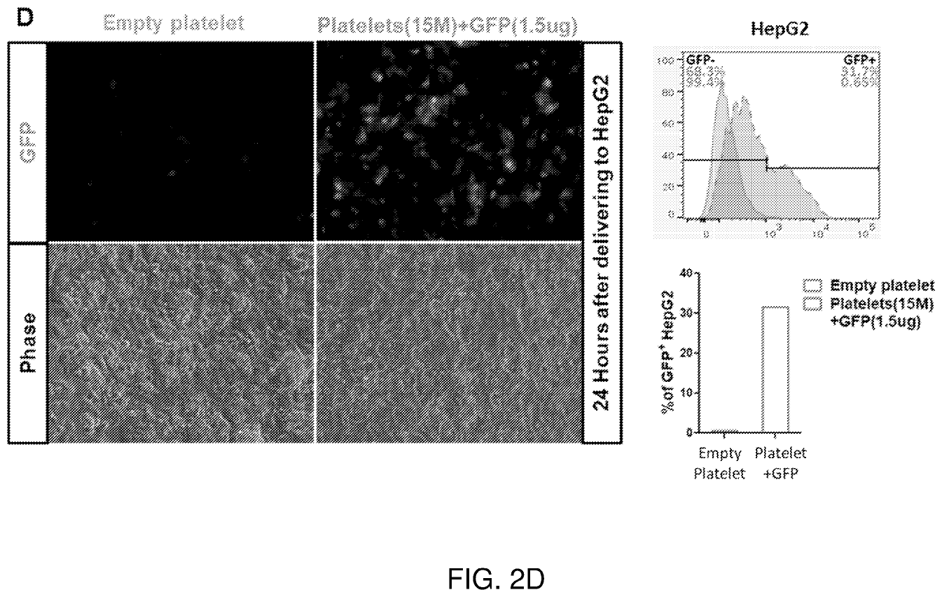

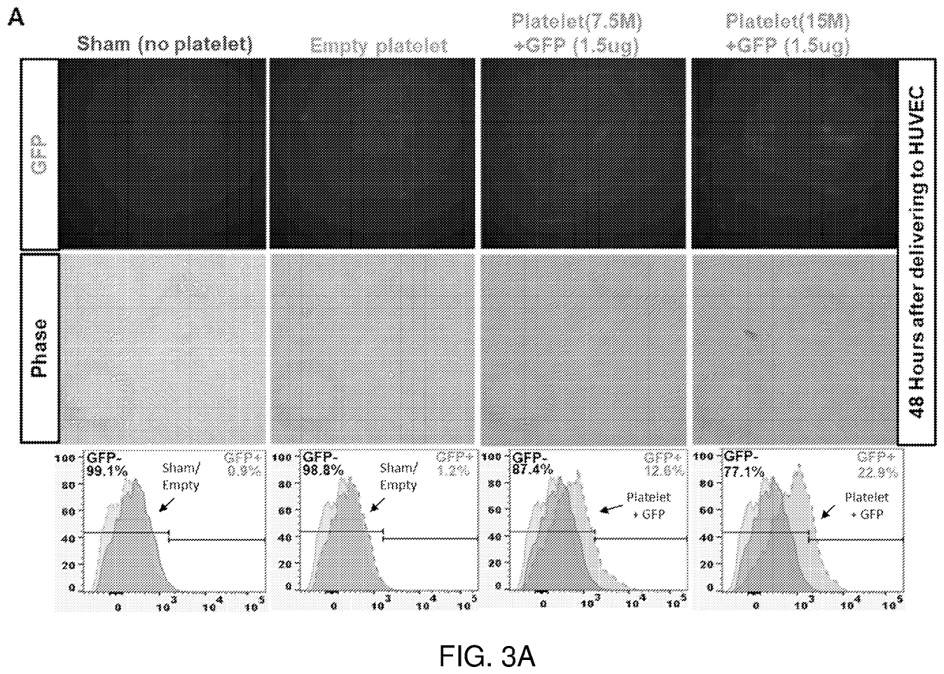

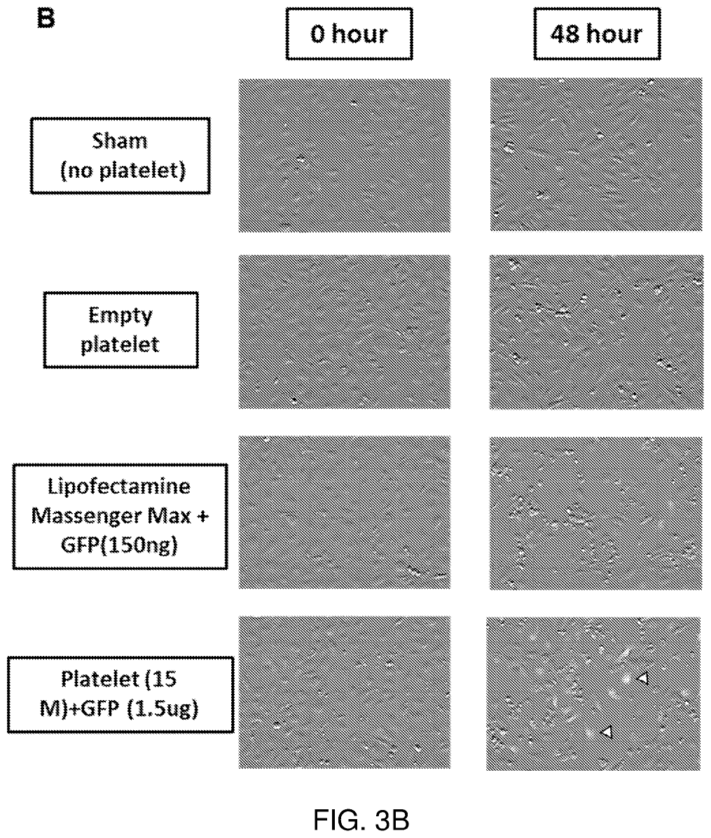

Using a functional GFP expression cassette as the mRNA cargo, expression was detected as early as 2 hours after platelet delivery (FIG. 2A-D, 3A-B). The expression peak was around 16-24 hours after platelet addition. Using a functional Firefly luciferase expression cassette as the mRNA cargo, luciferin was added 16 hours after delivery at a 150 μg/mL final concentration. Luminescent signal was quantified 15 minutes later.

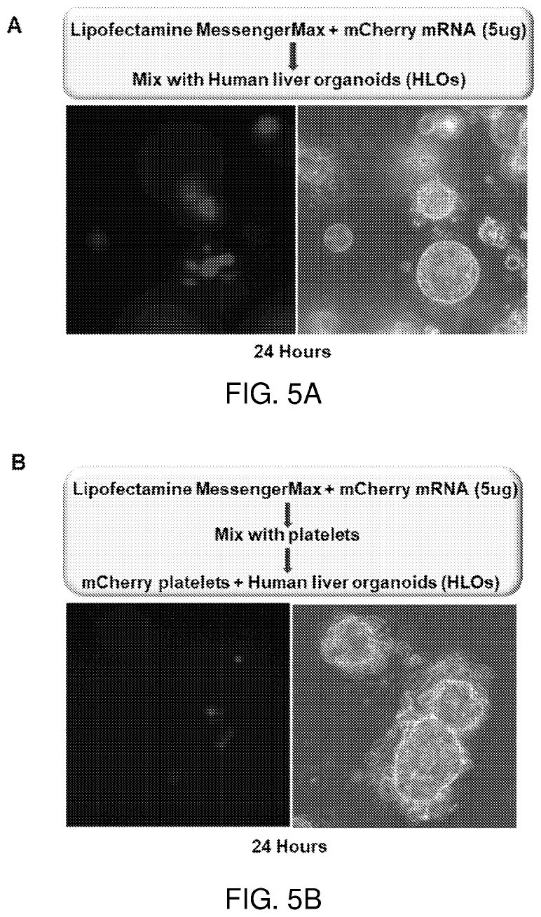

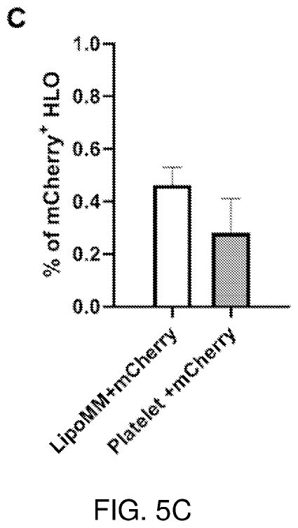

Using a functional mCherry expression cassette as the mRNA cargo, platelets were transfected and used to transform human liver organoids (HLOs). These HLOs transformed with mCherry-platelets showed successful mCherry fluorescence comparable to a Lipofectamine MessengerMAX control (FIG. 5A-B).

### Example 3. Platelet-Based Delivery of Proteins

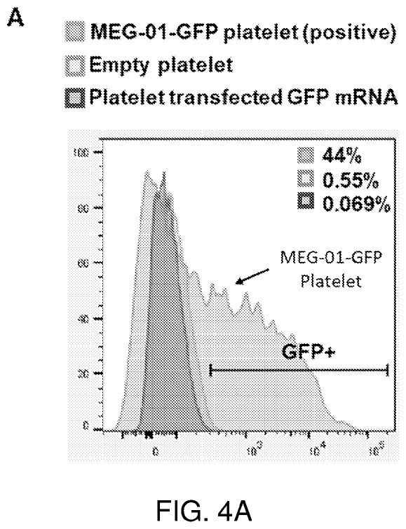

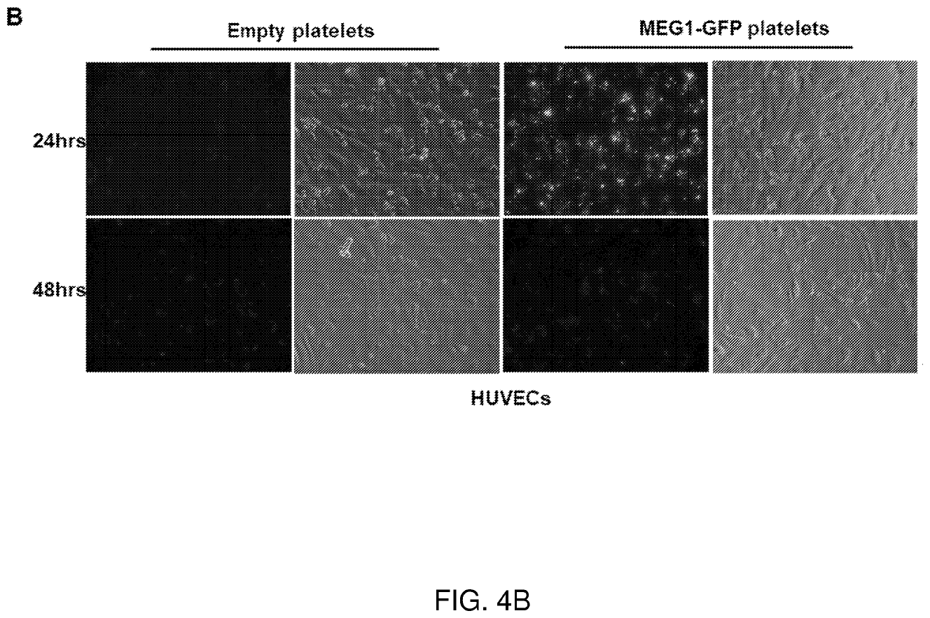

In addition to nucleic acids such as mRNA, modified platelets were used to deliver protein. MEG-01 megakaryocytes were transduced with pLV-GFP lentivirus to induce expression of GFP (MEG-01-GFP). These megakaryocytes were cultured to produce GFP-positive platelets (FIG. 4A), which were co-cultured with HUVECs. HUVECs showed GFP fluorescence after co-culture (FIG. 4B).

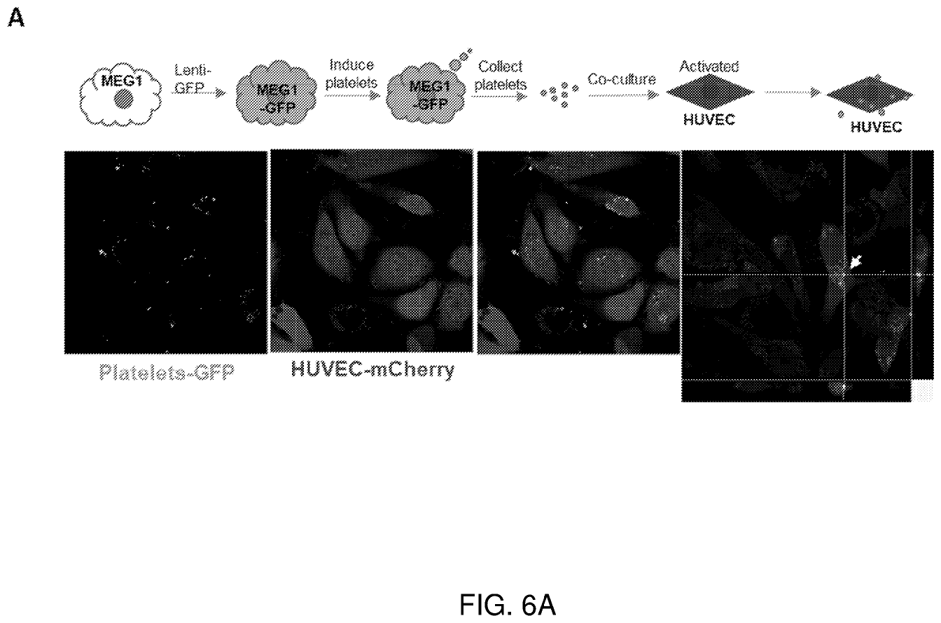

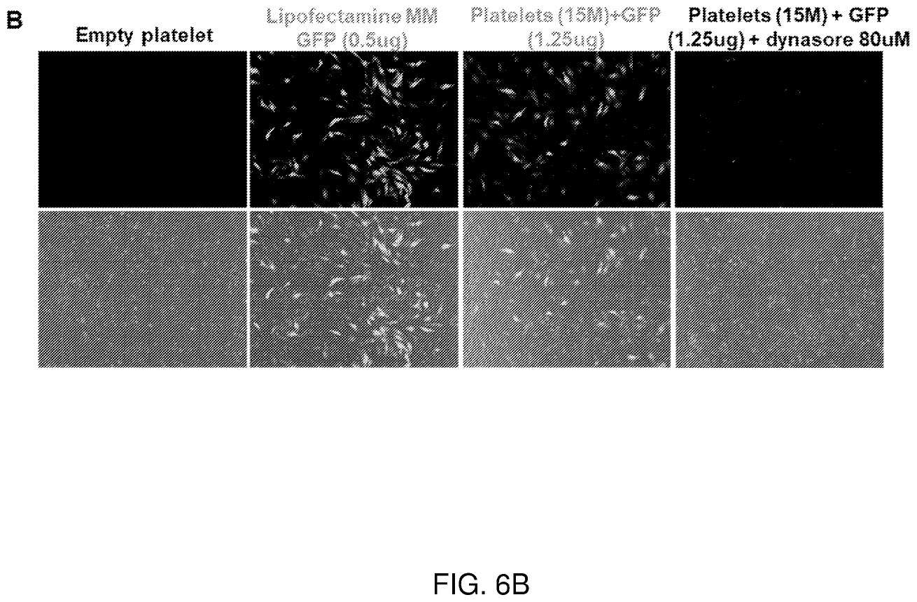

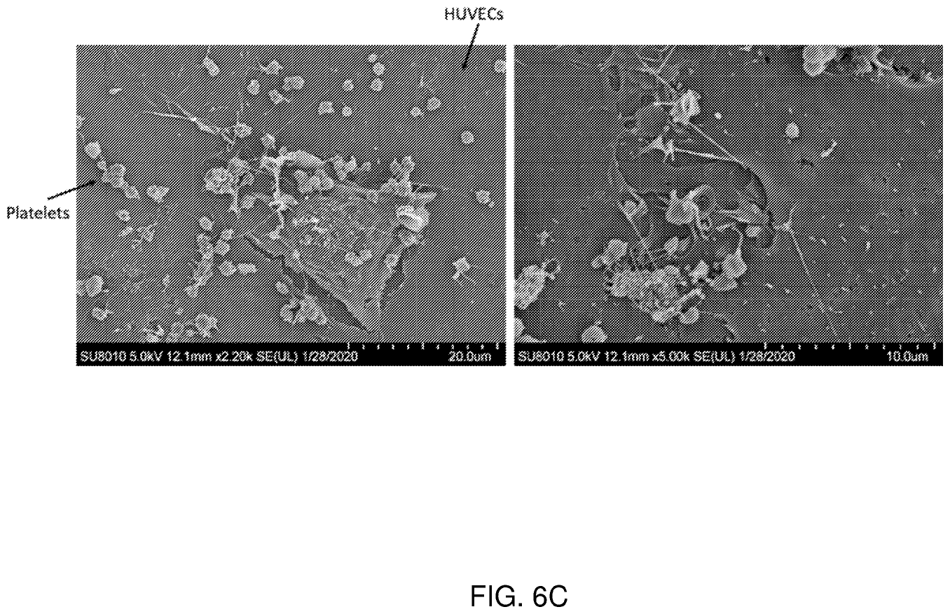

In a separate experiment, GFP-positive platelets were co-cultured with mCherry-expressing HUVECs. These co-cultured HUVECs exhibited dual GFP and mCherry fluorescence (FIG. 6A). Platelets treated with the dynamin inhibitor dynasore (at an 80 μM concentration) were unable to transform HUVECs (FIG. 6A). Electron microscopy showed the intimate interaction between platelets and HUVECs and filamentous structures suggestive of tunneling nanotubes (FIG. 6C).

### Example 4. In Vivo Delivery of Modified Platelets

6-12 week old NSG mice were prepared. Each mouse was to be injected with 10-20 μg of luciferase mRNA loaded into human platelets (scaling up the mRNA to platelet ratio as described in Example 1). After lipofection of the platelets with the mRNA, the mRNA/platelet transfection mixture was further prepared by centrifuging at 1800× at room temperature for 10 minutes, washing with EBM, and finally resuspending the platelets in 100-200 μL of 5% D-glucose solution. This platelet solution was loaded into an insulin syringe and injected into the tail vein of the NSG mice.

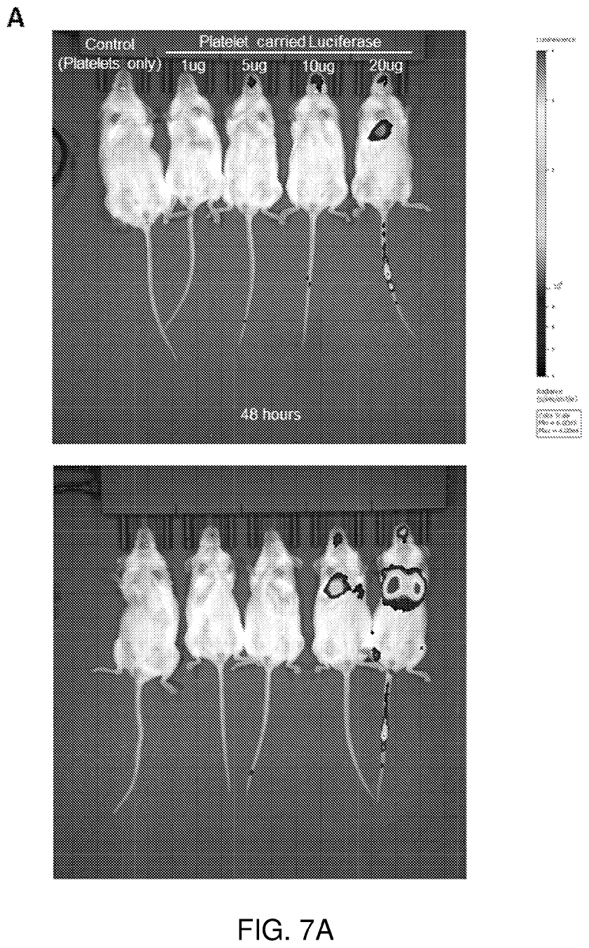

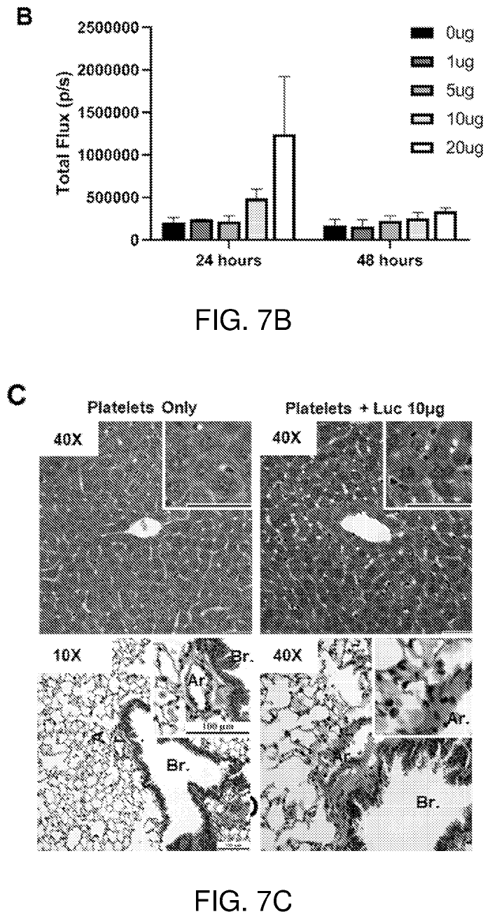

24 hours after platelet delivery, 2.25 mg of luciferin per mouse was injected peritoneally. Imaging of the whole mouse was performed, for example, with an IVIS Spectrum In Vivo Imaging System (PerkinElmer) (FIG. 7A-B). For harvesting of the individual murine organs, each mouse was perfused with 150 μg/mL luciferin in PBS through the right ventricle. The organs were harvested and imaged for luminescence or by hematoxylin/cosin staining (FIG. 7C).

### Example 5. Human Platelet-Based In Vivo Gene Delivery to Mouse Cells

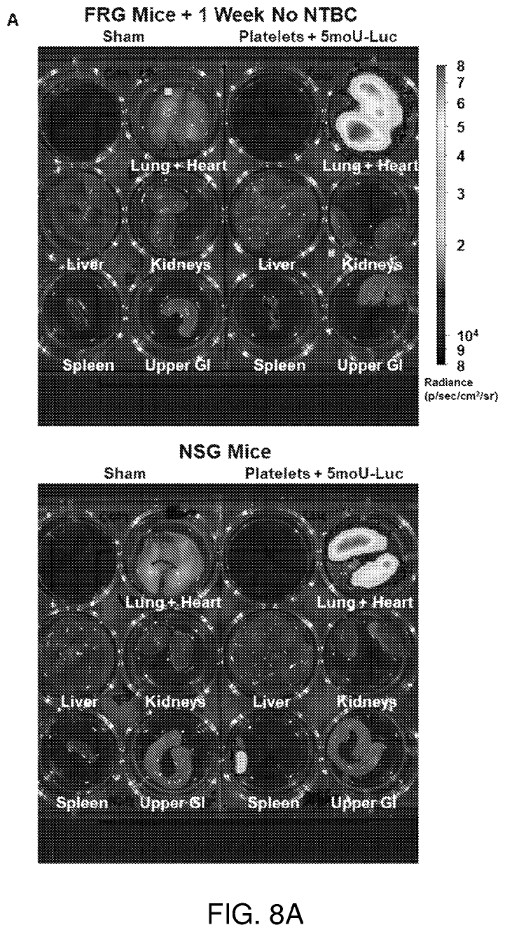

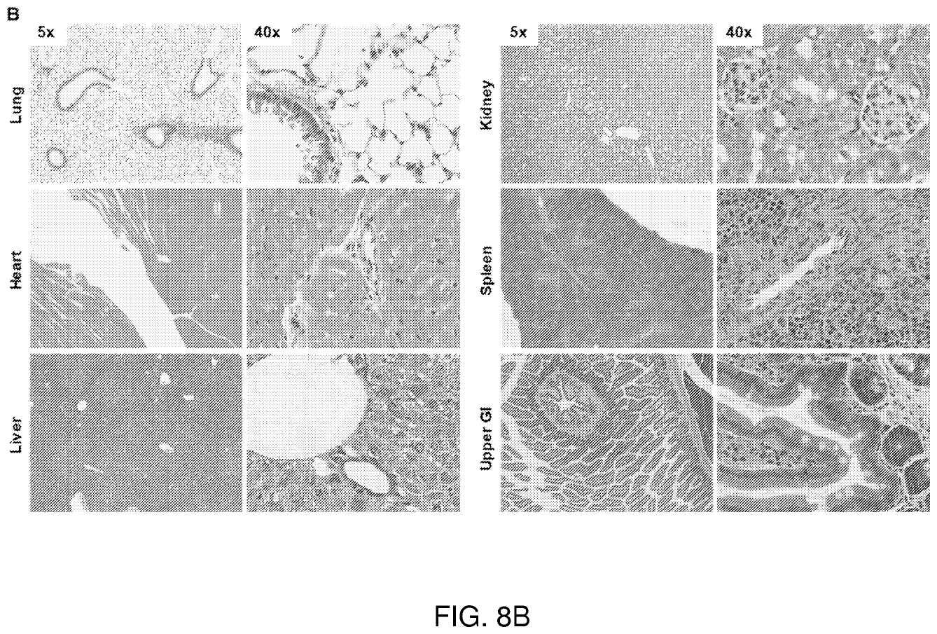

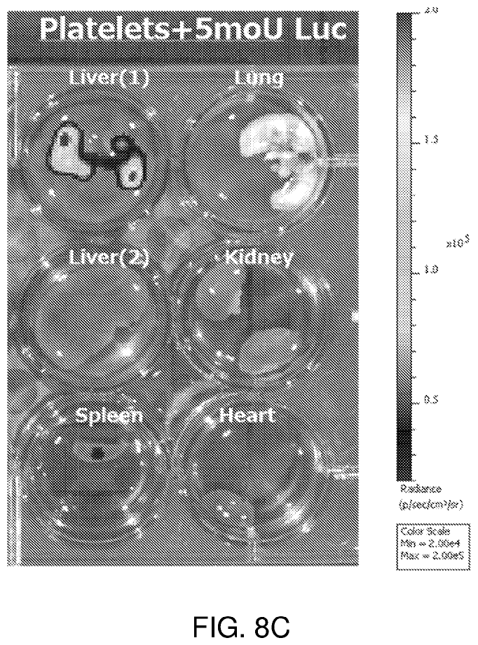

Platelets were loaded with 20 μg 5-moU stabilized luciferase mRNA using Lipofectamine MessengerMax (Thermo Fisher). The standard MessengerMax protocol was used to formulate lipoplexes using 32 μL of MessengerMax mixed with 20 μg of mRNA in 1.6 mL Opti-MEM (Thermo Fisher) for 10 minutes. Platelets were thawed at room temperature and washed once with EGM2 (Lonza) at room temperature with no supplements added (1800 g, 10 min). 200 million platelets were resuspended with 400 μL of EGM2 and mixed with the previously formulated MessengerMax+mRNA in Opti-MEM. mRNA loading was achieved during a 1 hour incubation at 37° C. Platelets were washed once in EGM2 (1800 g, 10 min) at room temperature, resuspended in 200 μL sterile normal (5%) D-glucose, and injected through the tail vein of FRG (Yecuris) or NSG mice (Jackson Laboratories). FRG mice were taken off 2-(2-nitro-4-trifluoromethylbenzoyl)-1,3-cyclohexanedione (NTBC) maintenance for 1 week prior to tail vein injection to induce liver damage phenotype. Sham controls were prepared in the same manner but without platelets (MessengerMax vector only). Luciferase expression was measured 24 hours post tail vein injection by luminescent imaging of excised organs following a 150 μL intraperitoneal injection of D-luciferin (15 mg/mL) in sterile PBS. Luminescent imaging reveals preferential luciferase mRNA transfer and expression in the lungs and heart for both FRG and NSG mice (FIG. 8A). NSG mice also exhibited luciferase expression in the spleen. Organs were maintained in a solution of D-luciferin (150 μg/mL) in PBS throughout the completion of imaging. Luminescence was quantified by the IVIS Spectrum In Vivo imaging system.

Organs of FRG mice with successful delivery of 200 million loaded with 20 μg luciferase mRNA imaged by IVIS were subsequently fixed in 4% paraformaldehyde, embedded in paraffin blocks, and sectioned at 5 microns. Tissue sections were stained with H&E prior to histological imaging (FIG. 8B).

To demonstrate feasibility of liver-targeting platelet-based in vivo gene delivery, platelets loaded with 20 μg of 5-moU stabilized luciferase mRNA were resuspended in 200 μL sterile PBS and injected into the spleen of FRG mice. Luciferase expression was measured 24hours post tail vein injection by luminescent imaging of excised organs following a 150 μL intraperitoneal injection of D-luciferin (15 mg/mL) in sterile PBS. Organs were maintained in a solution of D-luciferin (150 μg/mL) in PBS throughout the completion of imaging. Luminescence was quantified by the IVIS Spectrum In Vivo imaging system. Luminescent imaging revealed preferential luciferase mRNA transfer and expression in the liver of FRG mice (FIG. 8C).

### Example 6. Human Platelet-Based In Vitro Gene Delivery to HEK293T Cells

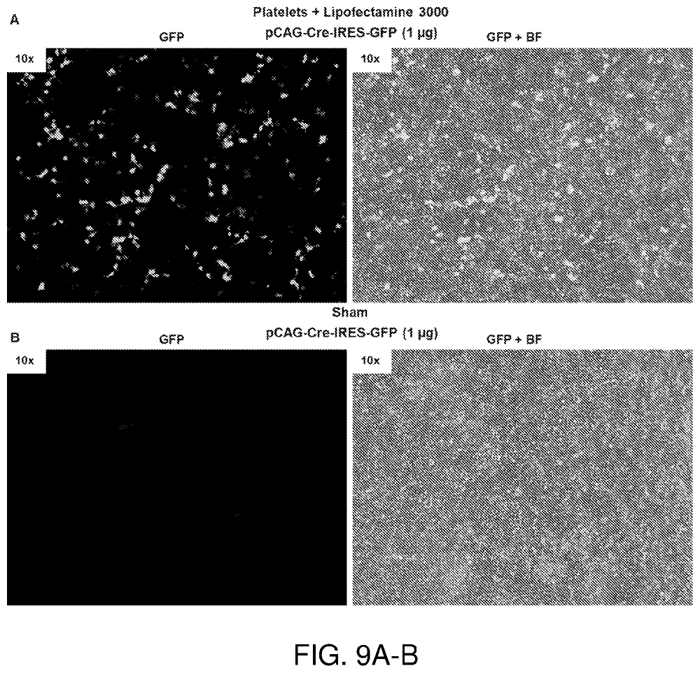

Platelets were loaded with 1 μg pCAG-Cre-IRES-GFP plasmid (Addgene) using Lipofectamine 3000 (Thermo Fisher). The standard Lipofectamine 3000 protocol was used to formulate lipoplexes using 2 μL P3000 reagent and 1.5 μL Lipofectamine 3000 in 100 μL Opti-MEM for 10 minutes. Platelets were thawed at room temperature. 10 million platelets were directly added to the formulated lipoplexes. Plasmid loading was achieved during a 1 hour incubation at 37° C. Platelets were pelleted at 1600 g for 10 minutes, resuspended in culture medium with thrombin to a final concentration of 0.1 U/mL, and added to HEK293T cells. Sham controls were prepared in the same manner but without platelets (Lipofectamine vector with plasmid). Expression of GFP in the HEK293T cells was observed for the ones treated with loaded platelets but not the sham control (FIG. 9A-B).

### Example 7. Human Platelet-Based Gene Delivery to Human Cells and Organoids

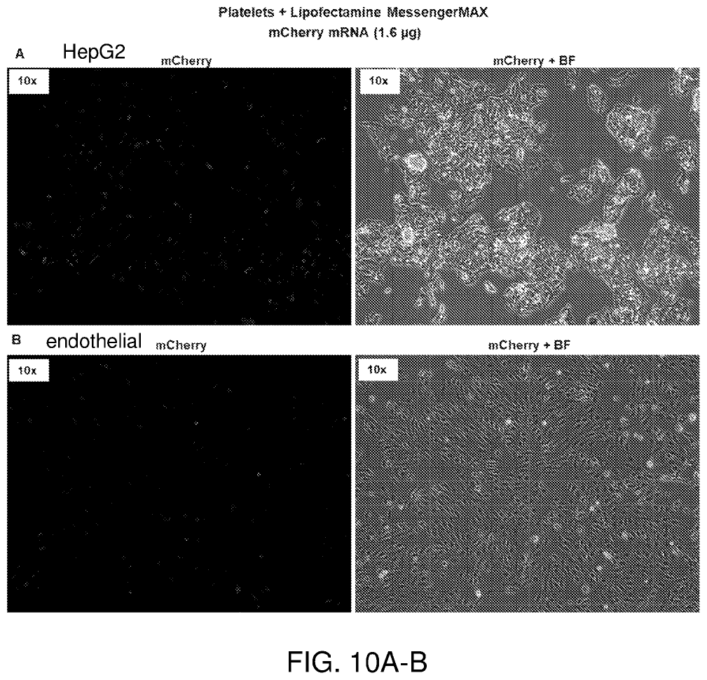

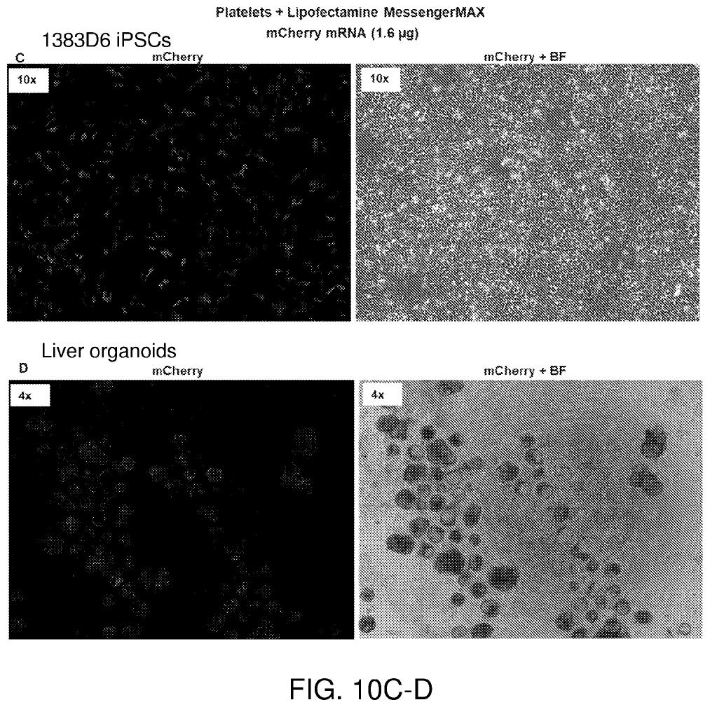

Platelet-based gene delivery was demonstrated in various human cell types. The cell types tested were HepG2 hepatocellular carcinoma, endothelial cells differentiated from human iPSCs, human iPSC line 1383D6, and liver organoids differentiated from human iPSCs. mCherry mRNA was transcribed from a PCR template using the HiScribe T7 ARCA mRNA kit with tailing (New England Biolabs). Platelets were loaded with mCherry mRNA using Lipofectamine MessengerMax. The standard MessengerMax protocol was used to formulate lipoplexes using 8 μL of MessengerMax mixed with 5 μg of mRNA in 400 μL Opti-MEM for 10 minutes. Platelets were thawed at room temperature and washed once with EGM2 (Lonza) at room temperature with no supplements added (1800 g, 10 minutes). Platelets were resuspended with 400 μL of EGM2 and mixed with the previously formulated MessengerMax+mRNA in Opti-MEM. mRNA loading was achieved during a 1 hour incubation at 37° C. Platelets were washed once in EGM2 (1800 g, 10 minutes) at room temperature, resuspended in 100 μL of culture medium. Then, 32 μL of platelet/mRNA resuspension (to deliver 1.6 μg mRNA) was added to the cells in a total volume of 1 mL culture media. After 24 hour incubation at 37° C., fluorescent images were obtained with a BZ-X810 fluorescence microscope (Keyence) using TRITC filter. Fluorescence images show that platelet-mediated gene delivery can be performed in different cell types including HepG2 (FIG. 10A), endothelial cells (FIG. 10B), iPSCs (FIG. 10C), and liver organoids (FIG. 10D).

### Example 8. Mouse Platelet-Based Gene Delivery to Human iPSC Cells

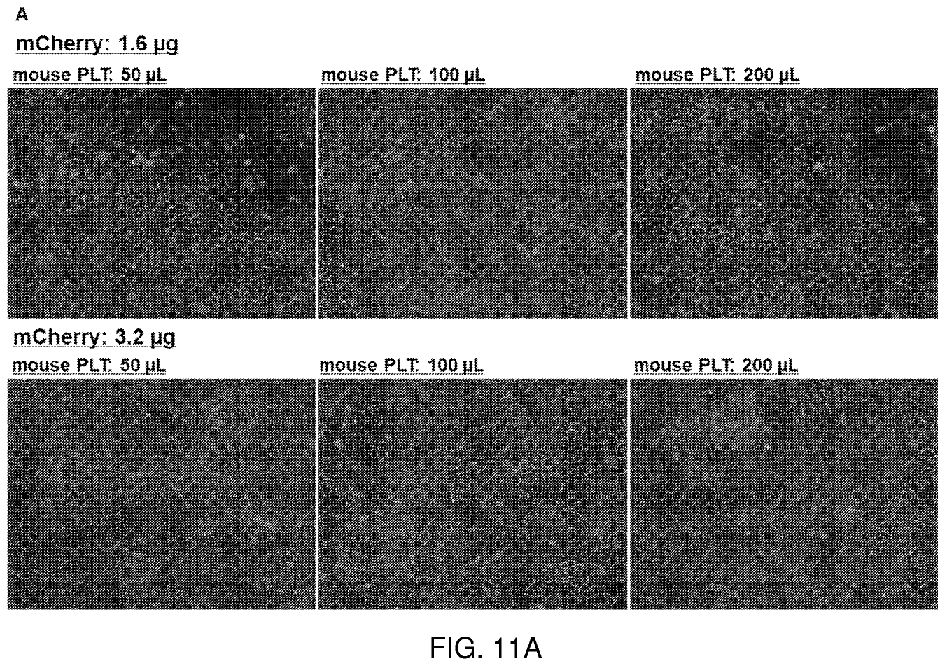

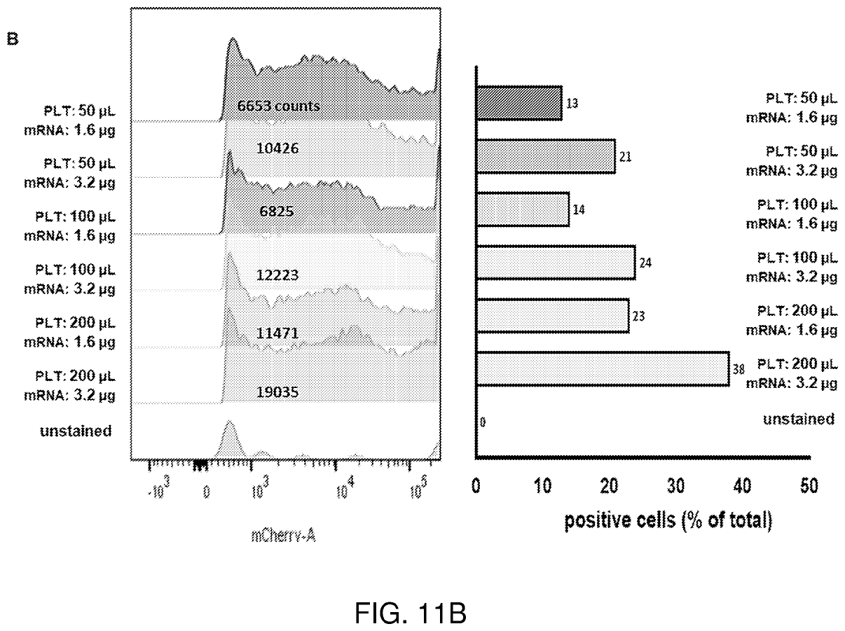

Mouse platelets were purified from 200 μL of blood using 16% iohexol gradient medium. The collected platelets (resuspended in either 50, 100, or 200 μL) were loaded with 10 μg mCherry mRNA using Lipofectamine MessengerMax. The mRNA loaded platelets were prepared using the same procedures as described in Example 7, followed by adding platelet/mRNA resuspension corresponding to either 1.6 μg or 3.2 μg mRNA to human iPSCs (clone 1383D6) in a total volume of 1 mL culture media. After 24- or 48-hour incubation at 37° C., fluorescent images were obtained using a BZ-X810 fluorescence microscope (Keyence) using TRITC filter. mCherry fluorescence shows that mouse platelets can be used to deliver genes to human iPSCs (FIG. 11A).

Subsequently, the transformed iPSCs were dissociated with 0.25% trypsin-EDTA solution and collected by centrifugation. The cells were washed once with PBS containing 3% FBS. The samples were then filtered through 0.22 μm filters and subjected to flow cytometric analysis on a FACSCanto I flow cytometer (BD Biosciences). Each of the platelet/mRNA conditions resulted in mCherry expression in the iPSCs, with increased expression generally observed with higher platelet count and mRNA amount (FIG. 11B).

### Example 9. Platelet Surface Modification to Enhance Cell Type Selectivity

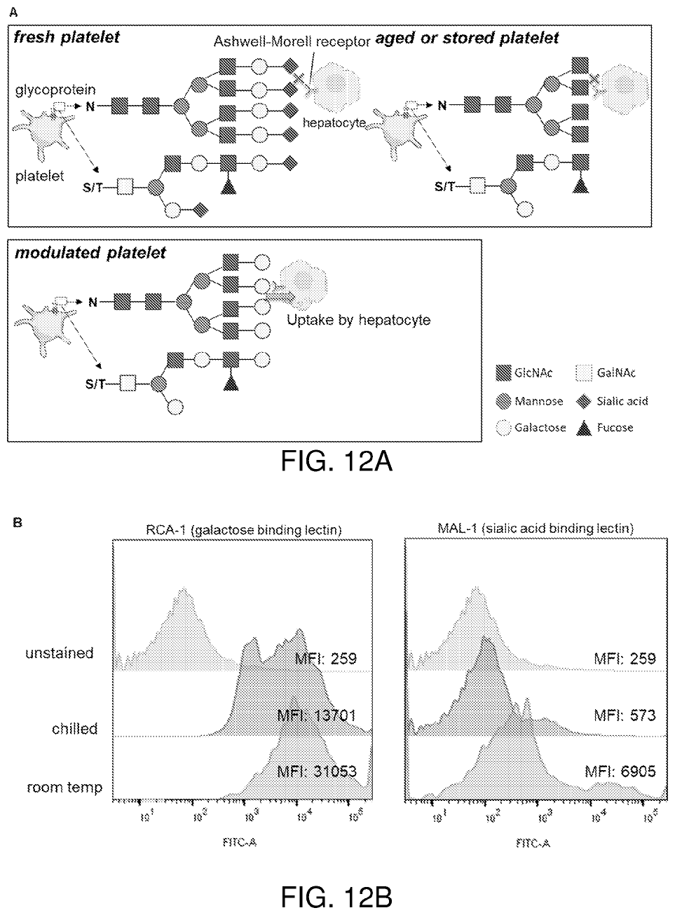

FIG. 12A depicts an exemplary schematic for improving selectivity of platelet-based gene delivery to hepatocytes. To maintain circulating platelet homeostasis, surface glycans of older platelets are desialylated through action of intrinsic sialidases stored in platelet lysosomes, which are released in response to senescent signals. The Ashwell-Morell receptor (AMR) expressed on hepatocytes are specific for terminal galactoses of platelet glycans exposed by desialylation, resulting in hepatic uptake and clearance of the platelet. Similarly, platelets stored in chilled conditions have been found to exhibit GlcNAc-exposed surface glycans, altering clearance by hepatic macrophages.

Fresh, room temperature stored platelets and chilled platelets were incubated with Ricinus communis agglutinin 1 (RCA-1; a galactose binding lectin) or Maackia amurensis lectin 1 (MAL-1; a sialic acid binding lectin) conjugated to FITC. Flow cytometry quantification of galactose and sialic acid suggests that chilled platelets are more frequently desialylated than room temperature platelets (FIG. 12B).

To take advantage of this, platelets or megakaryocytes are modified to display modified surface glycans lacking sialic acid in order to improve uptake to hepatocytes. Modification can be achieved, for example, by incubating platelets or megakaryocytes with sialidases or neuraminidases prior to mRNA loading and/or platelet administration. It is envisioned that this can be used to deliver genetic material for the treatment of hepatic diseases and abrogating undesired platelet-mediated delivery to other cell types.

### Example 10. Delivery of Gene Editing Complexes Using Platelets

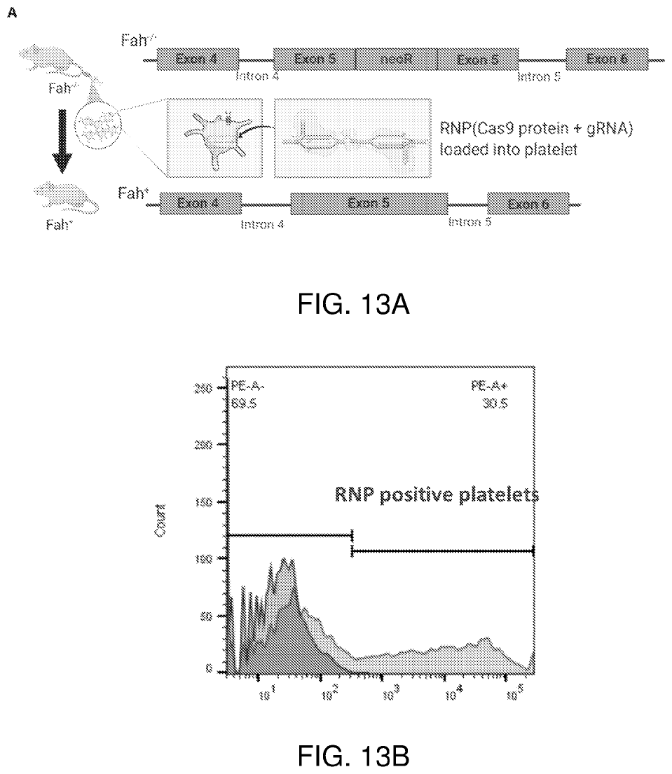

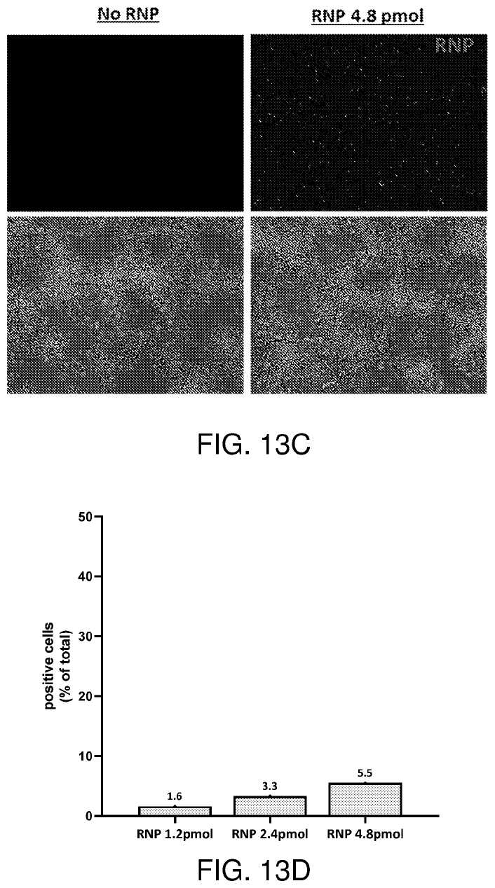

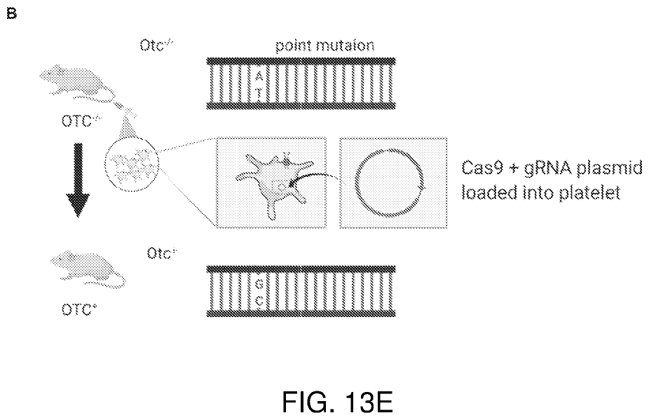

Tyrosinemia type 1 is an inborn error of tyrosine catabolismcaused by defective activity of fumarylacetoacetate hydrolase (Fah). An animal model of tyrosinemia type 1 is achieved by the insertion of a neomycin resistance cassette within exon 5 of the Fah genetic sequence. Platelets loaded with a gene editing system such as CRISPR/Cas9 specific for exon 5 of Fah can be delivered to the tyrosinemia type 1 animal model, removing the inserted neoR and restoring Fah function (FIG. 13A).

To demonstrate feasibility of platelet-based gene-editing complex delivery, fluorescently labeled tracrRNA that can monitor delivery of CRISPR/Cas9 complex was used. Human iPSC line 1383D6 were used as exemplary target cells. The cells were plated at a density of 2×10⁵ cells/well in a 12 well plate and grown overnight at 37° C. with 5% CO₂. Two crRNAs (crRNA1, 5′-UCUACUCUUCUCGGCAGUCG-3′ (SEQ ID NO: 1); crRNA2, 5′-GUGGUCGAGGCUAGAACUAG-3′ (SEQ ID NO: 2)) were designed targeting against the neomycin resistance cassette sequence (which would be inserted within Fah in the tyrosinemia type 1 animal model). A duplex of crRNA and tracrRNA-ATTO-550 (gRNA) was prepared in equimolar concentrations (1 μM) in IDT buffer (Integrated DNA Technologies) by incubating at 95° C. for 5 min. The ribonucleoprotein (RNP) complex was generated via the assembly of 9 μL of 1 μM recombinant Alt-R S. pyogenes HiFi Cas9 Nuclease V3 (Integrated DNA Technologies) with 9 μL of 1 μM gRNA and 3.6 μL of Cas9 PLUS Reagent in a total volume of 150 μL Opti-MEM (Thermo Fisher Scientific) by incubating at room temperature for 5 min. The RNA complex was mixed with 7.2 μL of Lipofectamine CRISPRMAX (Thermo Fisher Scientific) in a total volume of 300 μL Opti-MEM, and incubated at room temperature for 20 min.

Separately, platelets were washed with EBM and resuspended in fresh 600 μL Opti-MEM. The RNA complex and the platelets were mixed and incubated at 37° C. for 2 hours. The RNP-loaded platelets were washed with EBM and resuspended in fresh 750 μL culture media. Then, 100, 200, or 400 μL of the RNP loaded platelets (corresponding to 1.2, 2.4, or 4.8 pmol RNP/well, respectively) was added to the cells in a total volume of 1 mL culture media. The remaining 50 μL sample was subjected to flow cytometric analysis to assess the percentage of ATTO 550 positive platelets. After 24 hour incubation at 37° C. with 5% CO₂, the transfected cells were harvested and subjected to flow cytometric analysis to detect ATTO 550 on a FACSCanto I flow cytometer (BD Biosciences).

A population of RNP loaded platelets were positive for ATTO 550, indicating presence of the RNP in the platelets (FIG. 13B). After incubation of 1383D6 iPSC cells with the RNP loaded platelets, the presence of RNP in the cells were able to be detected by ATTO 550 fluorescence (FIG. 13C). This transfer of RNP to the iPSCs was dose-dependent according to the amount of RNP used (FIG. 13D).

Ornithine transcarbamylase (OTC) deficiency, one of the most common inborn errors of metabolism, is an inherited disorder that causes ammonia accumulation in the blood. Pathogenic variants include point mutations of the OTC gene resulting in dysfunctional protein. Platelet-mediated delivery of a gene editing system such as CRISPR/Cas9 specific for the OTC point mutation can be used to modify the point mutation to restore normal OTC function (FIG. 13E).

### Example 11. Modification of Platelet Targeting by Ligand Anchoring

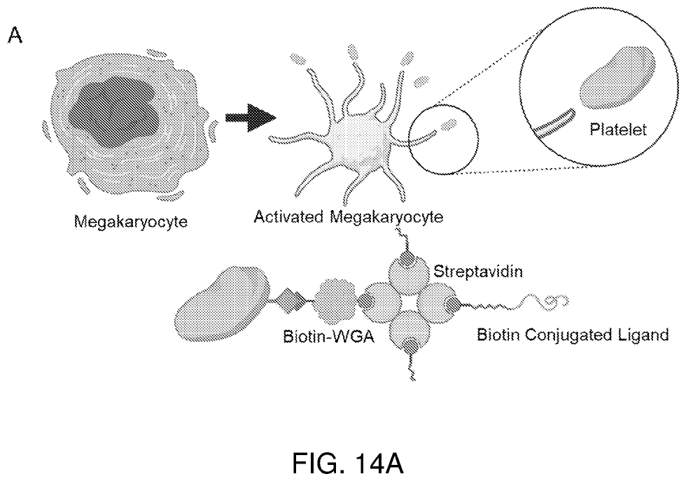

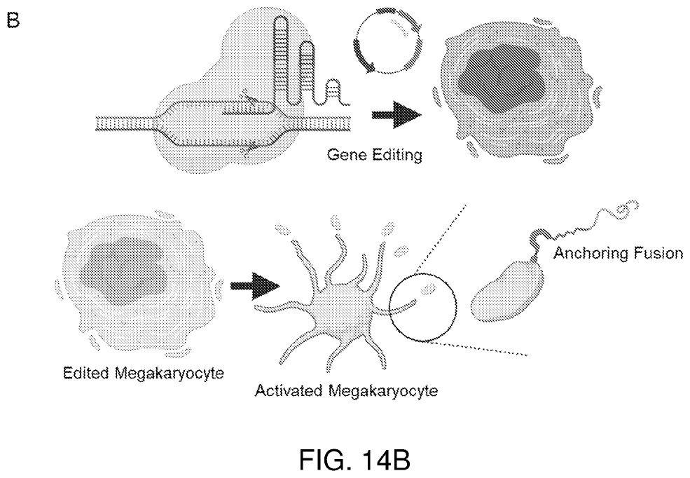

FIG. 14A depicts an exemplary schematic for anchoring desired targeting ligands to the surface of platelets, which improve specificity towards a certain cell type. Naturally occurring extracellular ligands on platelet surfaces bind to proteins such as wheat germ agglutinin (WGA). Using an approach involving biotinylated WGA bound to platelets, a biotinylated targeting ligand, and multimeric streptavidin to bring the WGA and targeting ligand together, resulting in platelets presenting the targeting ligand (FIG. 14A). As a non-limiting example, the targeting ligand may be O-GlcNAc, and presentation of O-GlcNAc through this approach can be used to modulate hepatic macrophage activity.

Another exemplary approach for anchoring desired targeting ligands may involve biotinylated Ricinus communis agglutinin (RCA1), which binds to galactose presented on platelets, where the biotinylated RCA1 and a biotinylated targeted ligand are brought together by multimeric streptavidin. The targeting ligand may be an asialoglycoprotein or a component thereof, which can induce ASGR1-mediated uptake into hepatocytes.

In an alternative method, megakaryocytes are genetically modified to knock-in an expression vector for a transmembrane cell surface targeting ligand. The targeting ligand is expressed and transferred to the formed platelets during megakaryocyte activation (FIG. 14B). It is envisioned that a wide range of transmembrane targeting ligands can be produced, either using naturally occurring transmembrane cell surface markers, or by fusing a transmembrane domain with an extracellular targeting domain (in either N-terminal or C-terminal orientation). These methods permit the preparation of platelets that exhibit specificity towards a certain cell type, for example, for the treatment of a disease manifested in these cell types.

### Example 12. Identification of Possible Anchoring Proteins

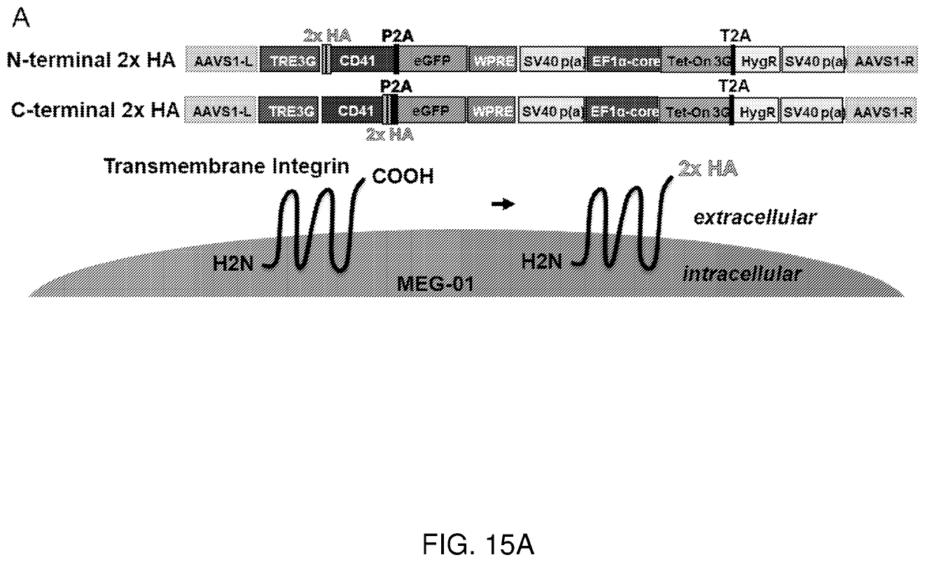

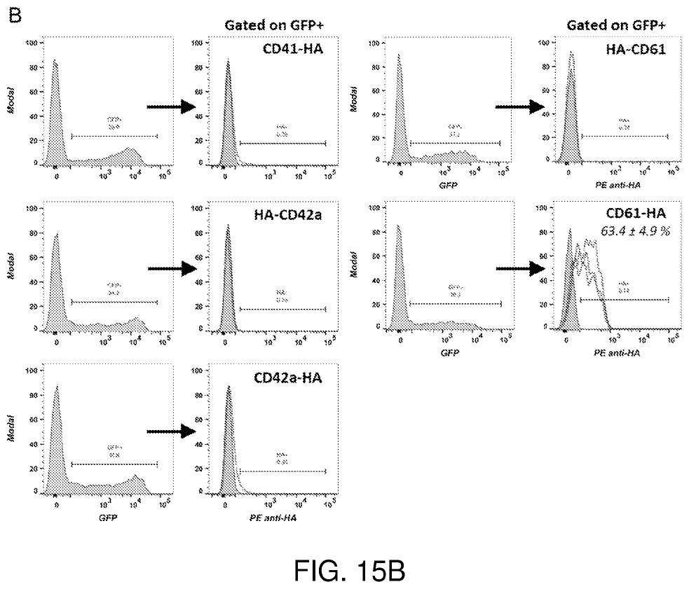

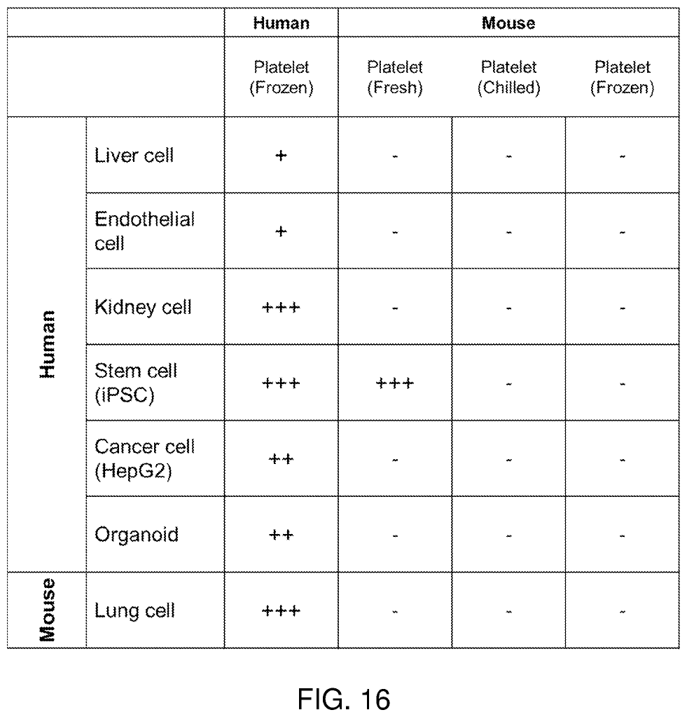

FIG. 15A depicts exemplary constructs used for the determination and identification of cell surface targeting ligands, which can be, for example, expressed on platelets to increase cell type specificity. The exemplary constructs involve custom TetOn expression vectors with left/right homologue arms to AAVS1. TetOn allows for conditional expression of targeting ligands or epitopes fused to the N- and C-termini of platelet cell surface markers (e.g. CD41, CD42a, CD61). Downstream cGFP through a P2A linker allows for successful identification of cells expressing the fusion vector. FIG. 15B depicts flow cytometry plots of 293T cells transfected with the exemplary N- or C-terminal fusion vectors depicted in FIG. 15A using CD41, CD42a, and CD61. Live cells were stained with a PE anti-HA fluorescent antibody and gated upon cGFP expression during flow cytometry analysis. PE fluorescence is shown against control (untransfected) 293T cells stained with PE anti-HA (shaded). The plots show that a C-terminal fusion of CD61 with an HA epitope results in robust exposure of the HA epitope on the 293T cell surface.

### Example 13. Protein Translation Efficiency of Transferred mRNA

FIG. 16 depicts different platelet and recipient cell types tested for platelet-mediated delivery, as analyzed by fluorescent protein translation efficiency from delivered mRNA.

### Example 14. Inhibitor of Tunneling Nanotubes Prevents Platelet-Based mRNA Delivery

The effects of cytochalasin B (CytoB), which is an inhibitor of tunneling nanotubes, on platelet-based mRNA delivery were investigated in the exemplary human iPSC line 1383D6. The cells were plated at a density of 2×10⁵ cells/well in a 12 well plate and grown overnight at 37° C. with 5% CO₂. Platelets were loaded with 5 μg of mCherry mRNA using Lipofectamine MessengerMax as described herein. The mRNA loaded platelets were further prepared by centrifuging at 1800×g at room temperature for 10 min, washing with EBM, and resuspending in fresh 600 μL culture media. Then, 100 μL of the mRNA loaded platelets (corresponding to 0.8 μg mRNA/well) containing 100 nM or 350 nM CytoB (or without CytoB as control) was added to the cells in a total volume of 1 mL culture media. After 24 hour incubation at 37° C. with 5% CO₂, fluorescent images were obtained with a BZ-X810 fluorescence microscope (Keyence) using a TRITC filter. Subsequently, the cells were harvested using 0.25% trypsin-EDTA solution, washed twice with PBS, and centrifuged. The cell pellet was dispersed using PBS containing 3% FBS, stained with LIVE/DEAD Fixable Green Dead Cell Stain Kit (Thermo Fisher Scientific) according to manufacturer's instructions. The cells were filtered through 0.22 μm filters and subjected to flow cytometric analysis on a FACSCanto I flow cytometer. Relative value in mean fluorescence intensity (MFI) was calculated with respect to the control samples without CytoB.

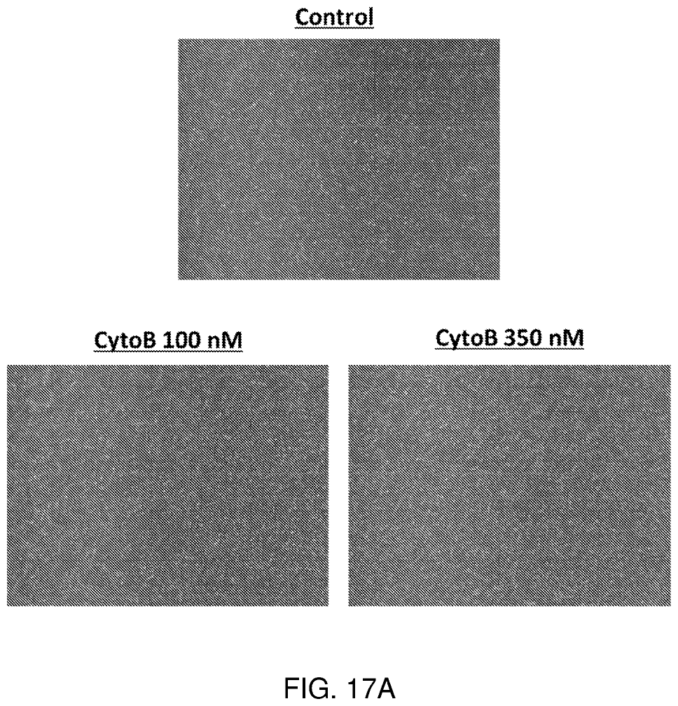

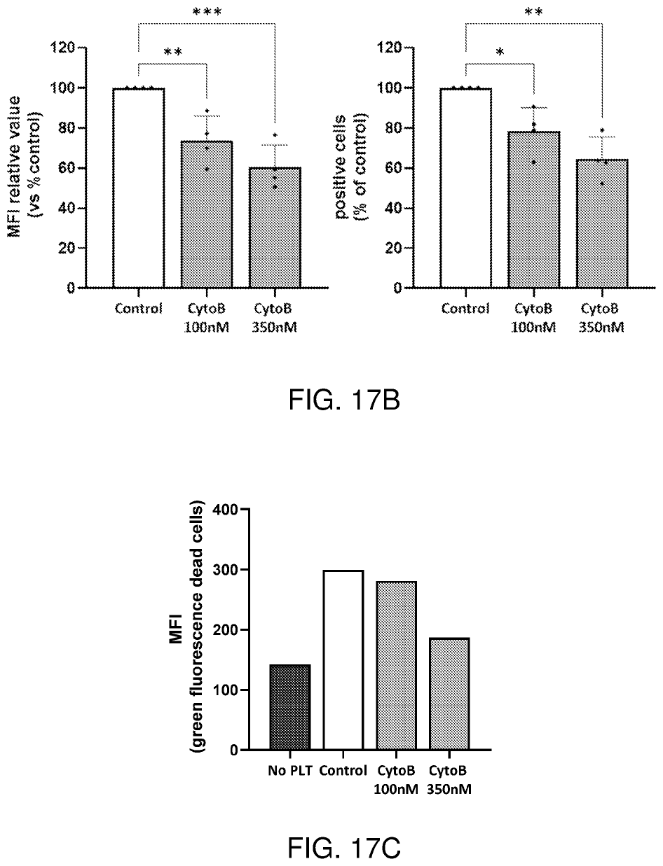

Representative fluorescence images detecting mCherry are depicted in FIG. 17A. FIG. 17B shows the MFI of mCherry fluorescence and percentage of mCherry-positive cells for iPSCs contacted with mRNA loaded platelets under 100 nM or 350 nM CytoB relative to control iPSCs contacted with mRNA loaded platelets without CytoB. FIG. 17C shows the MFI for green fluorescence indicating CytoB does not cause cell death for the conditions tested, with no CytoB control as well as control iPSCs that have not been contacted with platelets or Lipofectamine. Dead cells would show >10000 MFI on the plot.

### Example 15. Tracking of Platelet-Based mRNA Delivery

The mechanisms that contribute to platelet-based mRNA delivery were examined using fluorescent-labeled platelets in the exemplary human iPSC line 1383D6. Cells were plated at a density of 2×10⁵ cells in a 12 well plate and grown overnight at 37° C. with 5% CO₂. Platelets were loaded with 5 μg of mCherry mRNA using Lipofectamine MessengerMax as described herein. The mRNA loaded platelets were fluorescently labeled with PKH67 red fluorescent labeling kit (Sigma-Aldrich) according to manufacturer's instructions, followed by washing with EBM, and resuspended in fresh 900 μL culture media. The PKH67 stain generally labels cell membranes, such that the platelets will fluoresce red. Then, 50, 100, or 300 μL of the fluorescently labeled mRNA-loaded platelets (corresponding to 0.3, 0.6, or 1.6 μg mRNA/well, respectively) was added to the cells in a total volume of 1 mL culture media. After 24 hours of incubation at 37° C. with 5% CO₂, the transformed iPSCs were harvested and subjected to flow cytometric analysis.

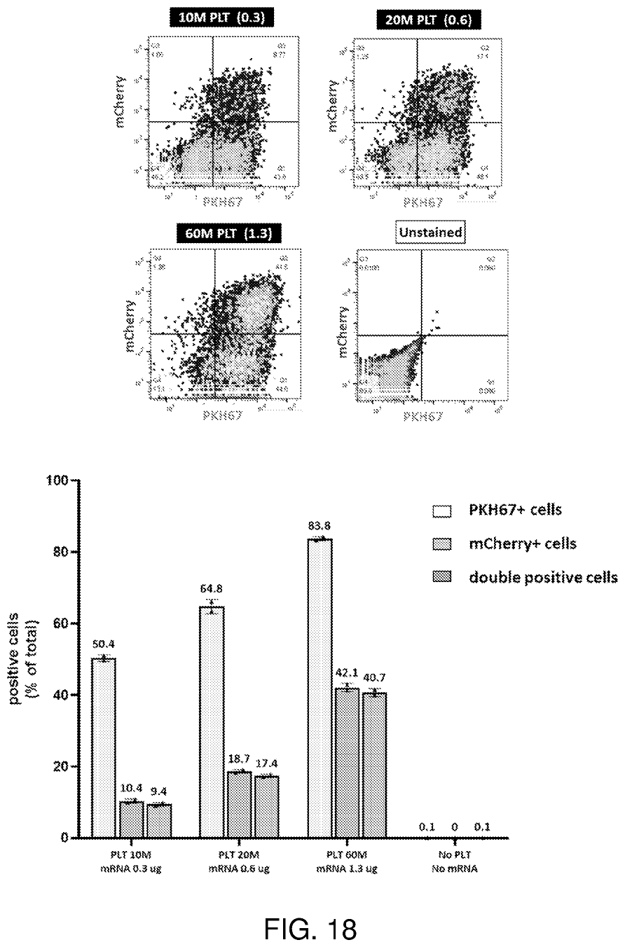

The population of PKH67+/mCherry+ double labeled iPSCs followed a dose-dependent manner respective to the amount of mRNA-loaded platelets added (FIG. 18), suggesting that platelet-derived contents can be delivered via endocytosis.

### Example 16. Platelet-Based Mitochondria Delivery

Human platelets were applied to mouse embryonic fibroblasts (MEFs) to determine whether platelet mitochondria could be delivered to the target cells. The cells were plated at a density of 5×10⁴ cells/well in a 12 well plate and grown overnight at 37° C. with 5% CO₂. Human platelets were prepared by centrifuging at 1800×g at room temperature for 10 min, washing with EBM, and resuspending in fresh 850 μL culture media. Then, 250 μL of the platelets (corresponding to 50 million cells/well) was added to the cells in a total volume of 1 mL culture media. After 24 hours of incubation at 37° C. with 5% CO₂, total cellular DNA was extracted using DNeasy Blood and Tissue Kit (Qiagen) and the quantity of total DNA was measured with a Qubit dsDNA HS Assay Kit (Thermo Fisher Scientific) according to manufacturer's instructions. Quantitative real-time PCR was performed in a reaction mixture containing 12.5 μL of PowerUp SYPR Green Master Mix (Thermo Fisher Scientific), 150 ng of extracted DNA, 1 μL each of 10 μM primer, adjusted to a total volume of 25 μL with nuclease-free water.

Cycling conditions were as follows: initial denaturation at 95° C. for 10 min, followed by 40 cycles of 95° C. for 15 sec and 62° C. for 60 sec. Primer sets used to amplify human or mouse nuclear DNA were as follows:

human mitochondrial tRNA-Leu^{(UUR)}:
forward,
(SEQ ID NO: 3)
5′-CACCCAAGAACAGGGTTTGT-3′,
reverse,
(SEQ ID NO: 4)
5′-TGGCCATGGGTATGTTGTTA-3′;
mouse nuclear 18s rRNA:
forward,
(SEQ ID NO: 5)
5′-TAGAGGGACAAGTGGCGTTC-3′,
reverse,
(SEQ ID NO: 6)
5′-CGCTGAGCCAGTCAGTGT-3′.

An aliquot of the cell samples was used to assess whether human platelet-derived mtDNA that was not delivered to MEFs remained in the sample of harvested cells. The cells in the sample were stained for 30 min with Alexa Fluor 700 anti-human platelet-specific CD41 antibody (BioLegend) and subjected to flow cytometric analysis.

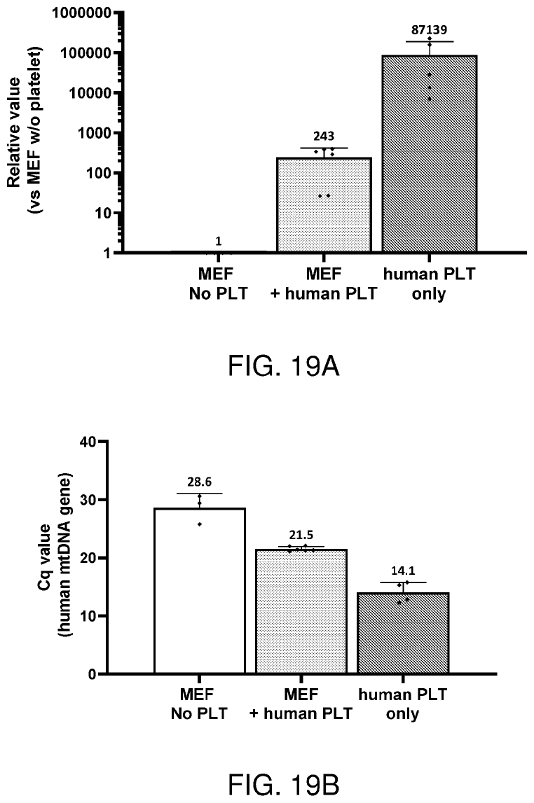

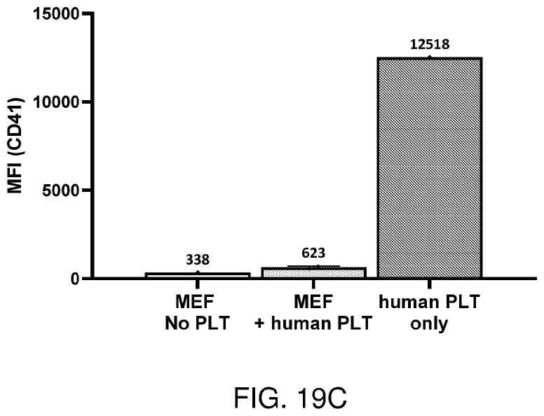

FIG. 19A shows the relative quantification of human mitochondrial DNA of MEFs contacted with human platelets and human platelets only, relative to MEF only (which do not amplify human mtDNA). FIG. 19B shows the quantification cycle (Cq) value for amplification of human mtDNA in MEFs contacted with human platelets and human platelets only, relative to MEF only. FIG. 19C shows MFI of human CD41 staining of MEFs contacted with human platelets, human platelets only, and MEF only control, indicating that the harvested cell sample did not contain significant residual platelets that were not taken up by the MEF cells and that the increase of amplified human mtDNA in MEFs contacted with human platelets are due to the human mitochondria delivered to the cells from the platelets.

### Example 17. In Vitro Differentiation of Umbilical Cord Blood-Derived or iPSC-Derived CD34+ Cells into Megakaryocytes and Platelets

To establish a more robust platelet-delivery system, in vitro differentiation of CD34+ cells into megakaryocytes and platelets was performed using the exemplary human iPSC line 1383D6. The cells were plated at a density of 200 cells/well on a laminin iMatrix-511-coated 6 well plate in Stem Fit Medium (Ajinomoto) supplemented with 10 μM Y-27632 ROCK inhibitor (day 0), and maintained at 37° C. in 5% CO₂. The medium was replaced every other day until day 7, where it was changed to Essential 8 Medium (Thermo Fisher Scientific) containing 80 ng/mL BMP4, 80 ng/mL VEGF, and 2 μM CHIR99021. After 48 hours (day 9), the medium was changed to Essential 6 Medium (Thermo Fisher Scientific) containing 20 ng/mL FGF2, 80 ng/mL VEGF, 50 ng/mL SCF, and 2 μM SB431542. After 48 hours (day 11), the medium was changed to StemPro 34 SFM Medium (Thermo Fisher Scientific) containing 10 ng/mL Flt-3L, 50 ng/mL VEGF, 50 ng/mL SCF, and 50 ng/mL TPO. After 48 hours (day 13), the medium was changed to StemPro 34 SFM Medium containing 5 ng/ml IL-11, 10 ng/mL Flt-3L, 10 ng/ml IL-3, 10 ng/mL IL-6, 25 ng/ml IGF, 50 ng/mL SCF, 50 ng/ml TPO, and 2 U/mL EPO. After 48 hours (day 15), the cells were dissociated with TrypLE, washed with PBS, resuspended in 300 μL of PBS/0.5% BSA with 100 μL of CD34 mAb microbeads (Miltenyi) and 100 μL of Fc receptor (FcR)-blocking reagent (Miltenyi). After incubation for 30 min, microbead-labeled cells were washed, resuspended in PBS/0.5% BSA, and passed through a 40 μm cell filter. CD34+ cells were isolated by MACS cell sorting system using MidiMACS Separator (Miltenyi), and then plated at a density of 2000 cells/well on a 96 well plate in HemaTox Megakaryocyte Medium containing HemaTox Megakaryocyte 100× Supplement (StemCell Technologies). The cells were cultured to be differentiated into mature megakaryocytes and platelets at 37° C. in 5% CO₂. The cultured cells were harvested at different time points and checked by light microscopy and flow cytometric analysis staining with Alexa Fluor 700 anti-human CD41 antibody.

Similarly, differentiation of human umbilical cord blood (UCB)-derived CD34+ cells into megakaryocytes and platelets were performed to compare the differentiation efficiency.

The platelets differentiated from UCB-derived CD34+ cells were used to demonstrate feasibility of platelet-based gene-editing complex delivery. The RNP complex was generated via the assembly of 13.5 μL each of 1 μM recombinant Alt-R S. pyogenes HiFi Cas9 Nuclease V3 and 1 μM gRNA with 5.4 μL of Cas9 PLUS Reagent in a total volume of 225 μL Opti-MEM by incubating at room temperature for 5 min. The RNP complex was mixed with 14.4 μL of Lipofectamine CRISPRMAX in a total volume of 450 μL Opti-MEM, and incubated at room temperature for 20 min. Then, 100 μL of the RNP complex (corresponding to 3 pmol RNP) was added to the cultured cells differentiated from UCB-derived CD34+ cells. After 24-hour incubation at 37° C. with 5% CO₂, the transfected cells were harvested, then washed with EBM and resuspended in fresh 1000 μL culture media. A 100 μL aliquot of sample was subjected to flow cytometric analysis to assess the percentage of ATTO 550 positive platelets. The RNP-loaded platelets were added to human iPSC line 1383D6, which were used as exemplary target cells, plated at a density of 2×10⁵ cells in a 12 well plate and grown overnight at 37° C. with 5% CO₂. After 24-hour incubation at 37° C. with 5% CO₂, the transfected cells were harvested and subjected to flow cytometric analysis staining with Alexa Fluor 700 anti-human CD41 antibody.

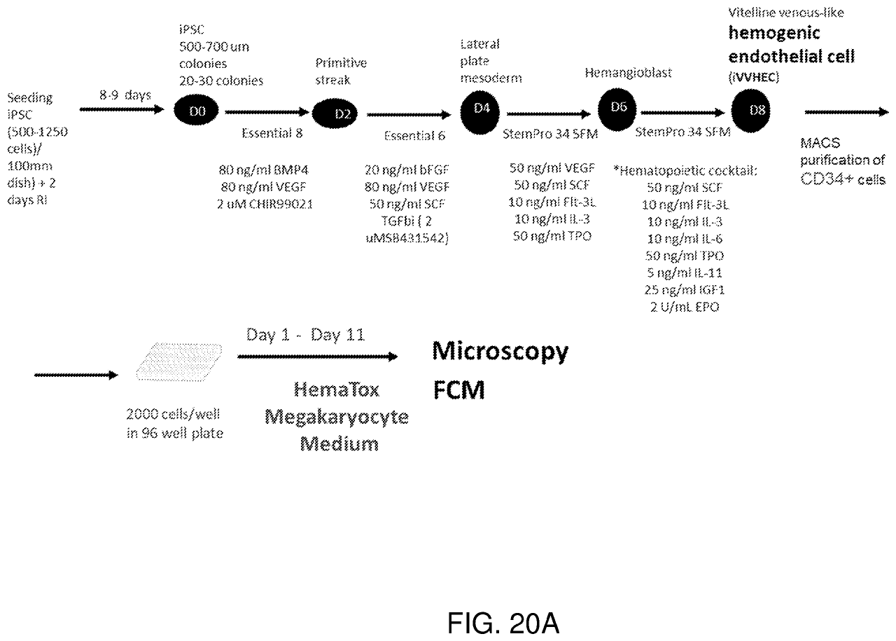

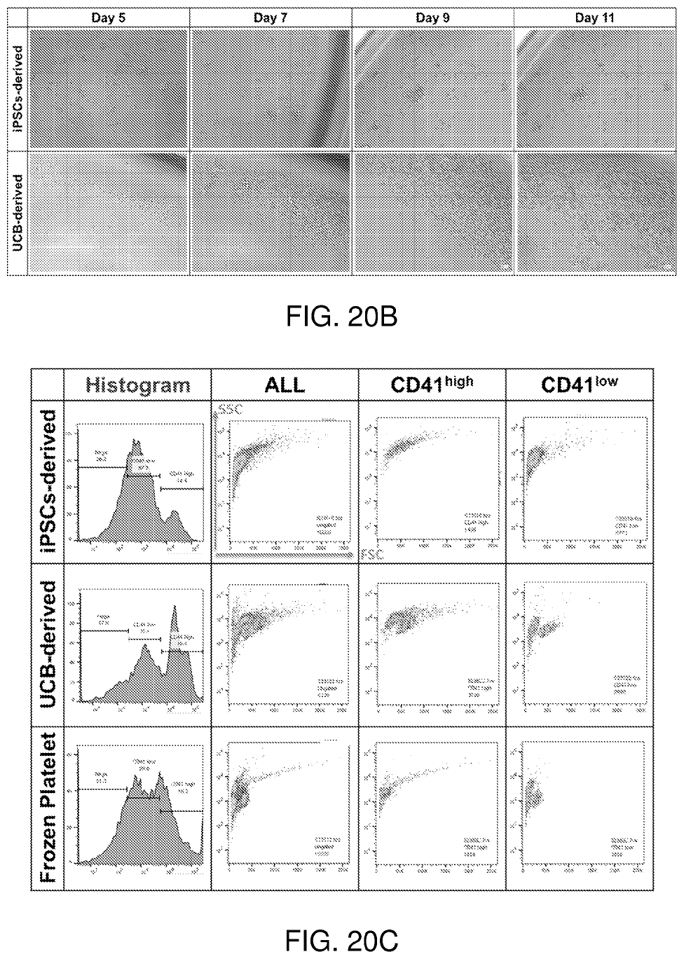

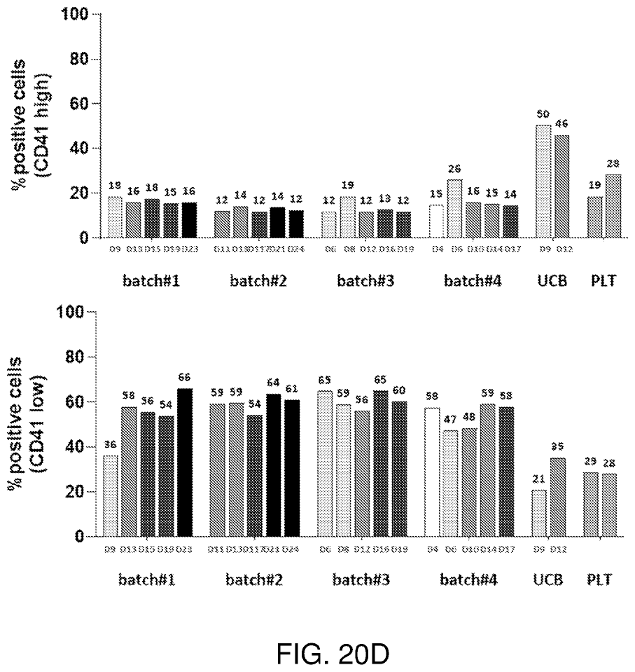

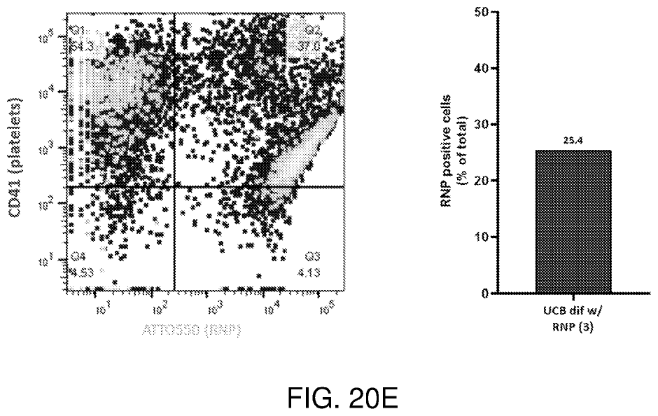

FIG. 20A shows an exemplary schematic for megakaryocyte/platelet differentiation. FIG. 20B shows light microscope images of iPSC and UCB derived CD34+ cells undergoing the differentiation process provided herein. FIG. 20C shows flow cytometric plots showing presence of CD41 positive cells differentiated from iPSCs and UCB CD34+ cells, compared to frozen platelets. FIG. 20D shows CD41 staining quantification of 4 different batches of iPSC-differentiated megakaryocyte/platelets, UCB-differentiated megakaryocyte/platelets, and frozen platelet controls. FIG. 20E shows a population of CD41positive/RNP positive cells in RNP-loaded UCB-derived platelets and 1383D6 iPSCs contacted with the RNP-loaded UCB-derived platelets. The RNP-loaded UCB-derived platelets showed higher transfection efficiency than primary human platelets transfected with RNP complex (e.g., as depicted in FIG. 13D).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the disclosed subject matter.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting.

All references cited herein, including but not limited to published and unpublished applications, patents, and literature references, are incorporated herein by reference for the subject matter referenced, and in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Blair T A et al. Mass Cytometry Reveals Distinct Platelet Subtypes in Healthy Subjects and Novel Alterations in Surface Glycoproteins in Glanzmann Thrombasthenia. Sci. Rep. (2018); 8:10300.
Dawkins H J S et al. Progress in Rare Diseases Research 2010-2016: An IRDiRC Perspective. Clin. Transl. Sci. (2018);11(1):11-20.
Hoffmeister K M, et al. Desialylated Platelets: A Missing Link in Hepatic Thromopoietin Regulation. The Hematologist. (2015);12(3).
Hoffmeister K M. The role of lectins and glycans in platelet clearance. J. Thromb. Haemost. (2011);9(s1):35-43.
Kaiser J. Virus used in gene therapies may pose cancer risk, dog study hints. (2020). doi:10.1126/science.aba7696.
Kowalski P S et al. Delivering the Messenger: Advances in Technologies for Therapeutic mRNA delivery. Mol. Ther. (2019);27(4):710-728.
Ohashi T. Enzyme Replacement Therapy for Lysosomal Storage Diseases. Pediatr. Endocrinol. Rev. (2012);10:26-34.
Ohmori T et al. Efficient expression of a transgene in platelets using simian immunodeficiency virus-based vector harboring glycoprotein Ibα promoter: in vivo model for platelet-targeting gene therapy. FASEB J. (2006);20(9):1522-4.
Ohta R et al. Hemogenic Endothelium Differentiation from Human Pluripotent Stem Cells in A Feeder- and Xeno-free Defined Condition. J. Vis. Exp. (2019); 148, e59823.
Perdomo et al. “Megakaryocyte Differentiation and Platelet Formation from Human Cord Blood-derived CD34+ Cells” J. Vis. Exp. (2017); 130, 56420.
Van Hoecke L & Roose K. How mRNA Therapeutics Are Entering the Monoclonal Antibody Field. J. Transl. Med. (2019);17(1):54.
Verdera H C et al. AAV Vector Immunogenicity in Humans: A Long Journey to Successful Gene Transfer. (2020);28(3):723-746.

What is claimed is:

1. A method of preparing a platelet comprising exogenous material and/or a derivative thereof, comprising:
a) contacting the platelet with the exogenous material and/or the derivative thereof, or
b) contacting a megakaryocyte with the exogenous material and/or the derivative thereof and allowing the megakaryocyte to produce the platelet comprising the exogenous material and/or the derivative thereof;
thereby preparing the platelet comprising the exogenous material and/or the derivative thereof.

2. The method of claim 1, wherein the exogenous material and/or the derivative thereof is biological material.

3. The method of claim 1 or 2, wherein the exogenous material and/or the derivative thereof comprises a fluid, a salt, a nutrient, a sugar, a small molecule, a lipid, an organelle, a mitochondrion, an endosome, a vesicle, a protein, a polypeptide, a peptide, an antibody, a nucleic acid, or any combination thereof.

4. The method of claim 3, wherein the small molecule is a therapeutic small molecule.

5. The method of claim 3 or 4, wherein the nucleic acid comprises DNA or RNA, or both.

6. The method of claim 5, wherein the RNA is messenger RNA (mRNA), noncoding RNA (ncRNA), antisense RNA (asRNA), long noncoding RNA (lncRNA), microRNA (miRNA), Piwi-interacting RNA (piRNA), small interfering RNA (siRNA), short hairpin RNA (shRNA), or guide RNA (gRNA), or any combination thereof.

7. The method of any one of claims 3-6, wherein the nucleic acid encodes for one or more viral, bacterial, or protozoal proteins or a portion thereof, or the protein is the one or more viral, bacterial, or protozoal proteins or portion thereof, wherein the one or more viral, bacterial, or protozoal proteins or portion thereof are immunogenic.

8. The method of any one of claims 3-6, wherein the nucleic acid encodes for a gene editing protein or the protein is the gene editing protein.

9. The method of claim 8, wherein the gene editing protein comprises a zinc finger nuclease, TALEN, nuclease, CRISPR nuclease, Cas9, or a base editor, and optionally, wherein the exogenous material and/or the derivative thereof further comprises a gRNA or a nucleic acid encoding for the gRNA.

10. The method of any one of claims 1-9, wherein contacting the platelet with the exogenous material and/or the derivative thereof or contacting the megakaryocyte with the exogenous material and/or the derivative thereof comprises transfection.

11. The method of claim 10, wherein the transfection comprises electroporation, lipofection, or viral transduction.

12. The method of any one of claims 1-11, wherein for option b):
i) the exogenous material and/or the derivative thereof comprised by the platelet is the exogenous material and/or the derivative thereof contacted with the megakaryocyte; or
ii) the exogenous material and/or the derivative thereof comprised by the platelet is derived from the exogenous material and/or the derivative thereof contacted with the megakaryocyte.

13. The method of claim 12, wherein the exogenous material and/or the derivative thereof comprised by the platelet comprises a protein and the exogenous material and/or the derivative thereof contacted with the megakaryocyte comprises a nucleic acid, wherein the protein is expressed from the nucleic acid.

14. The method of claim 12, wherein the exogenous material and/or the derivative thereof comprised by the platelet comprises a first nucleic acid and the exogenous material and/or the derivative thereof contacted with the megakaryocyte comprises a second nucleic acid, wherein the first nucleic acid is the second nucleic acid or is derived from the second nucleic acid.

15. The method of claim 12, wherein the exogenous material and/or the derivative thereof comprised by the platelet comprises a first protein and the exogenous material and/or the derivative thereof contacted with the megakaryocyte comprises a second protein, wherein the first protein is the second protein or a post-translationally modified variant of the second protein.

16. The method of any one of claims 1-15, wherein the platelet further comprises a cell surface modification that enhances selectivity to a specific recipient cell type or types.

17. The method of claim 16, wherein the cell surface modification comprises a modification to the surface glycans of the platelet.

18. The method of claim 16 or 17, wherein the cell surface modification comprises desialylation of the surface glycans of the platelet.

19. The method of any one of claims 16-18, wherein the cell surface modification comprises an exogenous targeting ligand on or associated with the cell surface of the platelet, wherein the exogenous targeting ligand selective for the specific recipient cell type or types.

20. The method of claim 19, wherein the exogenous targeting ligand is biotinylated and is bound to a streptavidin complex, which is further bound to a biotinylated platelet-specific ligand that is bound to the platelet.

21. The method of claim 19, wherein the exogenous targeting ligand comprises a transmembrane component and an extracellular component.

22. The method of claim 21, wherein the transmembrane component is a protein normally found in platelets.

23. The method of claim 22, wherein the protein normally found in platelets comprises CD41, CD42a, CD42b, CD61, CD9, CD29, CD31, CD36, CD62P, CD63, CD107a, CD154, glycoprotein VI, integrin αIIbβ3, or any combination thereof.

24. The method of any one of claims 19-23, wherein the exogenous targeting ligand is expressed by the platelet and/or the megakaryocyte from which the platelet is produced.

25. The method of any one of claims 1-24, further comprising stimulating the megakaryocyte with a megakaryocyte stimulator.

26. The method of claim 25, wherein the megakaryocyte stimulator is thrombopoietin, romiplostim, eltrombopag, avatrombopag, lusutrombopag, or any combination thereof.

27. The method of any one of claims 1-26, further comprising activating the platelet comprising the exogenous material and/or the derivative thereof with a platelet activator.

28. The method of claim 27, wherein the platelet activator is thrombin, ADP, calcium, thromboxane A2 (TXA2), platelet activating factor (PAF), cathepsin G, von Willebrand factor, collagen, fibrinogen, or laminin, or any combination thereof.

29. A method of delivering exogenous material and/or a derivative thereof to a recipient cell, comprising contacting a platelet comprising the exogenous material and/or the derivative thereof with the recipient cell, thereby delivering the exogenous material and/or the derivative thereof to the recipient cell.

30. The method of claim 29, wherein the platelet is produced by any one of methods of claims 1-28.

31. The method of claim 29 or 30, wherein the platelet comprises a gene editing protein and the gene editing protein modifies the genome of the recipient cell.

32. The method of claim 31, wherein the gene editing protein modifies the genome of a plurality of recipient cells, optionally wherein the plurality of recipient cells is part of an organ or tissue.

33. The method of any one of claims 29-32, wherein the recipient cell(s) is/are characterized by a disease and the modification to the genome of the recipient cell(s) treats the disease.

34. The method of claim 33, wherein the disease is tyrosinemia type 1 and the modification to the genome comprises a modification to the fumarylacetoacetate hydrolase (Fah) gene.

35. The method of claim 33, wherein the disease is ornithine transcarbamylase deficiency and the modification to the genome comprises a modification to the ornithine transcarbamylase (OTC) gene.

36. The method of claim 33, wherein the disease is citrullinemia type 1 (CTLN1) and the modification to the genome comprises a modification to the argininosuccinate synthetase (ASS1) gene.

37. The method of claim 33, wherein the disease is adult-onset type II citrullinemia (CTLN2) and the modification to the genome comprises a modification to the citrin (solute carrier family 25, member 13; SLC25A13) gene.

38. The method of any one of claims 29-37, wherein said contacting occurs in vitro.

39. The method of any one of claims 29-38, wherein said contacting occurs ex vivo.

40. The method of claim 39, wherein said recipient cell(s) are obtained or derived from a recipient subject, and further comprising administering the recipient cell(s) to the recipient subject following the step of contacting the platelet with the recipient cell(s).

41. The method of any one of claims 29-37, wherein said contacting occurs in vivo.

42. The method of claim 41, wherein said recipient cell(s) is/are in a recipient subject, and said contacting comprises administering said platelet to said recipient subject.

43. The method of claim 40 or 42, wherein the recipient subject is suffering from a disease and is in need of treatment, and wherein the exogenous material and/or the derivative thereof treats the disease.

44. The method of claim 42 or 43, wherein administration of the platelet to the recipient subject induces immune protection against a virus, bacteria, or protozoan in the recipient subject.

45. The method of any one of claims 42-44, wherein the platelet comprising the exogenous material and/or the derivative thereof is administered to the recipient subject parenterally, intramuscularly, intraperitoneally, intravenously, subcutaneously, trans-hepatically, intra-vascularly, intra-hepatically, intra-portally, intra-splenically, or intradermally, optionally by ultrasound-guided injection.

46. The method of any one of claims 29-45, wherein the platelet and/or megakaryocyte is obtained or derived from a donor subject, optionally wherein the platelet and/or megakaryocyte is obtained or derived from pluripotent stem cells obtained or derived from the donor subject.

47. The method of claim 46, wherein the platelet and/or megakaryocyte is obtained or derived from pluripotent stem cells obtained or derived from the donor subject according to a method comprising:
a) differentiating the pluripotent stem cells obtained or derived from the donor subject to hemogenic endoderm (HE);
b) differentiating the HE to CD34+ cells; and
c) differentiating the CD34+ cells to the megakaryocyte;
wherein the megakaryocyte produces the platelet.

48. The method of claim 46 or 47, wherein the donor subject has been provided with a megakaryocyte stimulator.

49. The method of claim 48, wherein the megakaryocyte stimulator is thrombopoietin, romiplostim, eltrombopag, avatrombopag, lusutrombopag, or any combination thereof.

50. The method of any one of claims 46-49, wherein the recipient subject or donor subject, or both, are mammals, optionally wherein the recipient subject and the donor subject are the same individual.

51. The method of claim 50, wherein the recipient subject or donor subject, or both, are human.

52. The method of any one of claims 29-51, wherein contacting the platelet with the recipient cell(s) is allogeneic or autologous.

53. A platelet prepared by the method of any one of claims 1-28.

54. The platelet of claim 53 for use in inducing immune protection against a virus, bacteria, or protozoan in a subject.

55. The platelet of claim 54 for use in editing a gene in a recipient cell.

56. The platelet for use of claim 55, wherein editing the gene in the recipient cell induces a desirable phenotype.

57. The platelet for use of claim 55 or 56, wherein editing the gene in the recipient cell treats a disease in the recipient cell.

58. The platelet for use of claim 57, wherein the disease is tyrosinemia type 1 and the edited gene is the Fah gene.

59. The platelet for use of claim 57, wherein the disease is ornithine transcarbamylase deficiency and the edited gene is the OTC gene.

60. The platelet for use of claim 57, wherein the disease is citrullinemia type 1 (CTLN1) and the edited gene is the argininosuccinate synthetase (ASS1) gene.

61. The platelet for use of claim 57, wherein the disease is adult-onset type II citrullinemia (CTLN2) and the edited gene is the citrin (solute carrier family 25, member 13; SLC25A13) gene.

62. The platelet for use of any one of claims 55-61, wherein the gene in the recipient cell is edited in vitro, ex vivo, or in vivo.

63. The platelet for use of any one of claims 55-62, wherein the platelet is administered to a recipient subject.

64. The platelet for use of claim 63, wherein the recipient subject is a mammal.

65. The platelet for use of claim 64, wherein the recipient subject is a human.

66. The platelet for use of any one of claims 63-65, wherein the platelet is obtained or derived from the recipient subject, optionally wherein the platelet is obtained or derived from a megakaryocyte obtained or derived from the recipient subject, optionally wherein the platelet and/or megakaryocyte is obtained or derived from pluripotent stem cells obtained or derived from the recipient subject.

67. The platelet for use of claim 66, wherein the platelet and/or megakaryocyte is obtained or derived from pluripotent stem cells obtained or derived from the recipient subject according to a method comprising:
a) differentiating the pluripotent stem cells obtained or derived from the recipient subject to hemogenic endoderm (HE);
b) differentiating the HE to CD34+ cells; and
c) differentiating the CD34+ cells to the megakaryocyte;
wherein the megakaryocyte produces the platelet.

68. A pharmaceutical composition comprising an effective amount of a platelet made by the method of any one of claims 1-28 and a pharmaceutically acceptable excipient, diluent, and/or carrier.
